SEQUENCE LISTING (19 sequences)

SEQ ID NO: 1 - protein, 14 aa, synthetic construct
LGKSGRLPGKSGRV

SEQ ID NO: 2 - protein, 14 aa, synthetic construct
AGKSGRLPGKSGRV

SEQ ID NO: 3 - protein, 14 aa, synthetic construct
LAKSGRLPGKSGRV

SEQ ID NO: 4 - protein, 14 aa, synthetic construct
LGASGRLPGKSGRV

SEQ ID NO: 5 - protein, 14 aa, synthetic construct
LGKAGRLPGKSGRV

SEQ ID NO: 6 - protein, 14 aa, synthetic construct
LGKSARLPGKSGRV

SEQ ID NO: 7 - protein, 14 aa, synthetic construct
LGKSGALPGKSGRV

SEQ ID NO: 8 - protein, 14 aa, synthetic construct
LGKSGRAPGKSGRV

SEQ ID NO: 9 - protein, 14 aa, synthetic construct
LGKSGRLAGKSGRV

SEQ ID NO: 10 - protein, 14 aa, synthetic construct
LGKSGRLPAKSGRV

SEQ ID NO: 11 - protein, 14 aa, synthetic construct
LGKSGRLPGASGRV

SEQ ID NO: 12 - protein, 14 aa, synthetic construct
LGKSGRLPGKAGRV

SEQ ID NO: 13 - protein, 14 aa, synthetic construct
LGKSGRLPGKSARV

SEQ ID NO: 14 - protein, 14 aa, synthetic construct
LGKSGRLPGKSGAV

SEQ ID NO: 15 - protein, 14 aa, synthetic construct
LGKSGRLPGKSGRA

SEQ ID NO: 16 - protein, 14 aa, synthetic construct
  SITE 3: note = nitroveratryloxycarbonyl-lysine
LGKSGRLPGKSGRV

SEQ ID NO: 17 - DNA, 74 bp, synthetic construct
  source 1..74: mol_type = other DNA
ttggttacgtcctcacttaggtgaggagaatggaaacgcaccattgaattatggttacttttaccattaaccaa

SEQ ID NO: 18 - DNA, 74 bp, synthetic construct
  source 1..74: mol_type = other DNA
tgcatacggtcctcacttaggtgaggagaatggaaacgcaccattattatggttacttttaccatgagtatgca

SEQ ID NO: 19 - DNA, 74 bp, synthetic construct
  source 1..74: mol_type = other DNA
aaaaaaaagtcctcacttaggtgaggagaatggaaacgcaccattaaaaaaaaaaaaaaaaaaaaaaaaaaaaa

(19) United States
(12) Patent Application Publication
(10) Pub. No.: US 2025/0186360 A1
(43) Pub. Date: Jun. 12, 2025

(54) LIQUID-LIQUID PHASE SEPARATION (LLPS)-BASED COMPARTMENTS COMPRISING A SHORT PEPTIDE AND AN ANIONIC POLYMER

(71) Applicant: RAMOT AT TEL AVIV UNIVERSITY LTD., Tel Aviv (IL)

(72) Inventors: Ayala LAMPEL, Tel Aviv (IL); Itai KATZIR, Tel Aviv (IL)

(21) Appl. No.: 19/045,829

(22) Filed: Feb. 5, 2025

Related U.S. Application Data
(63) Continuation of application No. PCT/IL2023/050797, filed on Aug. 1, 2023.
(60) Provisional application No. 63/398,673, filed on Aug. 17, 2022.

(51) Int. Cl. A61K 9/51; C12N 7/00

(52) U.S. Cl. CPC A61K 9/5169; C12N 7/00; C12N 2760/18422; C12N 2760/18423

(57) ABSTRACT

The present invention provides a composition comprising liquid-liquid phase separation (LLPS)-based compartments comprising a cationic peptide and an anionic polymer, variations and modifications thereof, methods for preparation of the LLPS-based compartments, and uses thereof for delivery or for controlling entrapping and releasing of a payload.

### CROSS REFERENCE TO RELATED APPLICATIONS

This application is a Bypass Continuation of PCT Patent Application No. PCT/IL2023/050797 having International filing date of Aug. 1, 2023, which claims the benefit of priority of U.S. Provisional Patent Application No. 63/398,673, filed Aug. 17, 2022, the contents of which are all incorporated herein by reference in their entirety.

### REFERENCE TO AN ELECTRONIC SEQUENCE LISTING

The contents of the electronic sequence listing (RMT-022-PCT.xml; Size: 18,200 bytes; and Date of Creation: Aug. 1, 2023) is herein incorporated by reference in its entirety.

### FIELD OF THE INVENTION

The present disclosure is generally directed to liquid-liquid phase separation (LLPS)-based compartments. Specifically, the invention relates to LLPS-based compartments based on peptides and an anionic polymer such as RNA for delivery of payloads.

### BACKGROUND OF THE INVENTION

Viral factories are membraneless intracellular compartments formed during the infection of various RNA viruses, including rabies, measles, and SARS-CoV-2. These compartments concentrate proteomic and genomic viral material and compartmentalize the replication and assembly of new viral particles. Recent studies suggest that viral factories, similar to other intracellular membraneless compartments including stress granules, nucleoli, and Cajal bodies, are dynamic and disordered, rather than solid hierarchical, assemblies formed by liquid-liquid phase separation (LLPS) of viral proteins and nucleic acids. Specifically, disordered regions within specific viral proteins play a role in complex coacervation and LLPS, where electrostatic interactions are thought to be the main driving force. Yet, the exact mechanisms of viral factories formation and the underlying network of intermolecular interactions vary between viral strains and are still not fully understood.

Such LLPS-based compartments formed by liquid-liquid phase separation may be useful for protection, sequestration, and delivery of molecules into cells. The advantage of these compartments is their dynamic nature which allows payloads to exchange with the surrounding environment and at the same time compartmentalize and regulate complex processes. So far, reported LLPS-based compartmentalization systems were prepared with intrinsically disordered proteins (IDPs). However, the construction of these systems involves a complex series of expression and purification steps, which produce limited yields, while the control over their physical and material properties remains a challenge. Accordingly, there is still a need for simple and controllable LLPS-based compartmentalization system.

### SUMMARY OF INVENTION

The following embodiments and aspects thereof are described and illustrated in conjunction with compositions and methods which are meant to be exemplary and illustrative, not limiting in scope. In various embodiments, one or more of the above-described problems have been reduced or eliminated, while other embodiments are directed to other advantages or improvements.

The present invention is directed to compositions comprising liquid-liquid phase separation (LLPS)-based compartments comprising a short peptide and an RNA, which may be used for encapsulating and releasing payloads. The present invention is further directed to the effect of changes in amino acid sequences and modifications of the properties of the compartments.

In some embodiments, there is provided a composition comprising a liquid-liquid phase separation (LLPS)-based compartment comprising a cationic peptide and an anionic polymer.

In some embodiments, the peptide has a length of about 5-20 amino acids.

In some embodiments, the peptide comprises at least one positively-charged amino acid. In some embodiments, the peptide comprises at least about 10% positively-charged amino acids. In some embodiments, the positively-charged amino acids are selected from lysine (Lys), arginine (Arg), and histidine (His).

In some embodiments, the peptide further comprises at least one hydrophobic amino acid and/or one aromatic amino acid. In some embodiments, the hydrophobic amino acid is selected from leucine (Leu), valine (Val), Alanine (Ala), isoleucine (Ile), and methionine (Met). In some embodiments, the aromatic amino acid is selected from phenylalanine (Phe), tyrosine (Tyr), Histidine (His), and tryptophan (Trp).

In some embodiments, the peptide further comprises glycine (Gly), proline (Pro), Serine (Ser), and/or threonine (Thr). In some embodiments, the peptide comprises the amino acids Arg or Lys, and Leu, Gly, Pro, Ser, and Val.

In some embodiments, the peptide comprises an amino acid sequence of a protein phosphate-binding loop (P-loop). In some embodiments, the peptide comprises an amino acid sequence of a P loop of a viral phosphoprotein (P protein). In some embodiments the peptide comprises an amino acid sequence of a P loop of a measles P protein.

In some embodiments, the peptide comprises a sequence selected from the sequences SEQ ID NOs: 1, 2, 3, 5, 6, 8, 9, 10, 12, 13, and 15. In some embodiments, the peptide comprises a sequence selected from the sequences SEQ ID NOs: 1, 6, 8, 9, 12, and 15. In some embodiments, the peptide comprises a sequence as set forth in SEQ ID NOs: 1.

In some embodiments, the peptide comprises at least one modified amino acid. In some embodiments, the at least one modified amino acid is modified by at least one cleavable group. In some embodiments, the at least one cleavable group is a hydrophobic and/or an aromatic cleavable group. In some embodiments, the at least one cleavable group is selected from a photocleavable group, a chemically cleavable group, and an enzymatically cleavable group. In some embodiments, the at least one cleavable group is a photocleavable group selected from ortho-nitrobenzyl (o-nitrobenzyl, ONB), o-nitroveratryloxycarbonyl (Nvoc), and a BODIPY (C9H7BN2F2 or 4,4-difluoro-4-bora-3a,4a-diaza-s-indacene)-derived photocleavable protecting group. In some embodiments, the at least one cleavable group is bound to a positively-charged amino acid. In some embodiments, the positively-charged amino acid is Lys.

In some embodiments, the peptide comprises a sequence according to SEQ ID NO: 16, including a modification of lysine at position 3 of SEQ ID NO: 1 by Nvoc.

In some embodiments, the anionic polymer is a nucleic acid. In some embodiments, the nucleic acid is RNA. In some embodiments, the anionic polymer comprises at least one secondary structure. In some embodiments, the peptide is modified by at least one cleavable group.

In some embodiments, the concentration of the peptide in the composition is at least 1 mM. In some embodiments, the concentration of the anionic polymer in the composition is at least 0.3 mg/ml. In some embodiments, the positive charges from the cationic peptide and the negative charges from the anionic polymer are at a ratio of at least about 2:1 positive/negative charges.

In some embodiments, the composition further comprises salt. In some embodiments, the peptide is modified by a cleavable aromatic group.

In some embodiments, the composition further comprises a payload. In some embodiments, the payload is hydrophilic. In some embodiments, the payload is hydrophobic. In some embodiments, the payload is selected from a protein and a nucleic acid. In some embodiments, the payload is an enzyme. In some embodiments, the payload has a size of about 500 Da to about 20 kDa.

In some embodiments, the compartment is suitable for releasing the payload following treatment with a light trigger, a chemical agent, or an enzyme.

In some embodiments, the compartment is suitable for releasing the payload following addition of salt, reduction of temperature, or a change of pH.

In some embodiments, the composition disclosed herein is provided for use in delivery of a payload to a cell.

In some embodiments, there is provided a method of preparing a liquid-liquid phase separation (LLPS)-based compartment, the method comprising mixing a cationic peptide, an anionic polymer, and optionally a payload.

In some embodiments, there is provided a method of releasing a payload from the LLPS-based compartment as disclosed herein, comprising treating the compartment with a light trigger, a chemical agent, an enzyme, salt addition, temperature reduction, or a pH change.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions.

### BRIEF DESCRIPTION OF DRAWINGS

The patent or application file contains at least one drawing executed in color. Copies of this patent or patent application publication with color drawing(s) will be provided by the Office upon request and payment of the necessary fee.

The invention will now be described in relation to certain examples and embodiments with reference to the following illustrative figures.

FIGS. 1A-1C show the design of LLPS-based compartments. FIG. 1A. Schematic illustration of viral factories 102 including a measles virus phosphoprotein (P protein) 104 formed in a host cell 106. A linear diagram of the P protein is presented on the right. The disordered P loop region of the P protein (108a, 108b) is indicated in the 3D schematic representation 104, and in the linear representation on the right, respectively. FIG. 1B. Chemical structure of viral factory-inspired peptide (VFP-1) containing 14 amino acids which are prevalent in the P protein P loop (LGKSGRLPGKSGRV—SEQ ID NO: 1). FIG. 1C. Top panel: chemical structure of Lys (position 3 of VFP-1) side chain modified by a photocleavable group Nvoc (left side). The Nvoc group is cleaved from the Lys side chain following UV irradiation (right side). Bottom panel, from left to right: schematics of liquid compartments formed by liquid-liquid phase separation (LLPS) of Nvoc-VFP-1 peptide and RNA (poly-U), encapsulating a fluorescent payload (110, red circle). Irradiation of the compartment leads to increased dynamics and payload release.

FIGS. 2A-2C show viral factory-inspired compartments formed by peptide/RNA LLPS. FIG. 2A. Phase diagram heatmap showing VFP-1/poly-U LLPS propensity at pH 7.5 measured by turbidity as a function of peptide/RNA concentration. FIGS. 2B-2C. Dynamic light scattering (DLS) (bottom panels) analyses of droplet size as a function of RNA (FIG. 2B) or peptide (FIG. 2C) concentration. FIG. 2B. Droplets formed by LLPS of 2 mM VFP-1 at varying poly-U concentrations (0.1 mg/ml, 0.3 mg/ml, 0.5 mg/ml). FIG. 2C. Droplets formed by 0.3 mg ml⁻¹ poly-U with VFP-1 at varying concentrations (1 mM, 2 mM, 3 mM). Scale bars=20 μm.

FIGS. 3A-3D show that electrostatic interactions are the main driving force of compartments formation as suggested by Ala scanning. FIG. 3A. Phase diagram heat map showing the effect of alanine substitution of each amino acid within VFP-1 (3 mM) on LLPS with poly-U at varying concentrations (0.3-1 mg/ml), as measured by turbidity as a function of RNA concentration. From top: VFP-1, and each one of amino acids 1-14 replaced with alanine. FIGS. 3B-3D. Fluorescence recovery after photobleaching (FRAP) analysis of VFP-1 and selected sequence variants (G5A, P8A, S11A, V14A) using laser scanning confocal microscopy with VFP-1-poly-U in the presence of Cy3-oligo-A. FIG. 3B. Confocal microscopy images of VFP-1 and the sequence variants at varying time points after photobleaching containing droplets formed by 3 mM peptide with 1 mg/ml poly-U. Scale bars=5 μm. FIG. 3C. Recovery plots as a function of time after photobleaching. FIG. 3D. Apparent t_{1/2} calculated from the recovery plots. Values represent average of n=9, standard deviation is presented.

FIGS. 4A-4E show compartment material properties of Nvoc-modified peptide compartments. FIG. 4A-4B. Effect of increasing NaCl concentration (FIG. 4A) or temperature (FIG. 4B) on formation of VFP-1 and Nvoc-VFP-1 droplets measured by a turbidity assay. FIGS. 4C-4E. FRAP analysis of compartments formed by Nvoc-VFP-1/poly-U at varying irradiation times showing tunable diffusivity. FIG. 4C. Confocal microscopy images of Nvoc-VFP-1 after varying irradiation time (0, 6, 18, 24 h) immediately, 3 sec, and 10 sec, after photobleaching. FIG. 4D. Recovery plots extracted from the FRAP analysis; and FIG. 4E. calculated t_{1/2}. Values represent average of n=10, standard deviation is presented. Scale bars=2 μm.

FIGS. 5A-5F. Light-triggered changes to the encapsulation efficiency of a payload (rhodamine B (RhB): FIGS. 5A-5C; Cyanine-5 (Cy5): FIGS. 5D-5F). FIGS. 5A, 5D. Encapsulation efficiency (EE) analysis of RhB or Cy5 in compartments formed by either VFP-1 or Nvoc-VFP-1 as a function of UV irradiation (t=24 h). FIGS. 5B-5C, 5E-5F. RhB or Cy5 release from VFP-1 and Nvoc-VFP-1 compartments following selective excitation (λₑₓ=405 nm) of individual droplets using laser scanning confocal microscopy. FIGS. 5B, 5E. RhB or Cy5 fluorescent signal in droplets (λₑₓ=561 nm) before and after irradiation of individual droplets. Values represent average of n=5, standard deviation is presented. FIGS. 5C, 5F. Confocal microscopy images of RhB release from droplets following irradiation. Scale bars=2 m.

### DETAILED DESCRIPTION OF THE INVENTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

Unless defined otherwise, all technical and scientific terms used herein have the same meaning as commonly understood by one of ordinary skill in the art to which the invention pertains.

The term “a” and “an” refers to one or to more than one (i.e., to at least one) of the grammatical object of the article. By way of example, “an element” means one element or more than one element.

The term “about” when referring to a measurable value such as an amount, a ratio, and the like, is meant to encompass variations of ±10% of the indicated value, as such variations are also suitable to perform the disclosed invention. Any numerical values appearing in the application are intended to be construed as if preceded by “about”, unless indicated otherwise.

Existing delivery vehicles, such as liposomes, lipid nanoparticles, polymersomes, DNA or protein, have several limitations, including, inter alia, size and charge limitation of payloads, and the need for an active, energy consuming, process for encapsulation.

In contrast, the liquid-liquid phase separation (LLPS)-based compartments of the invention have features which may overcome the above issues. As described in more detail below, the compartments of the present invention are membraneless (“open”) permeable compartments, allowing diffusion between the outer, low-density, phase and the inner, high-density, phase. Accordingly, under encapsulation-permissive conditions, a payload may easily be captured from the surrounding of the compartment by diffusion, and under release-permissive conditions the payload may be similarly easily released by diffusing out of the compartment. This allows a spontaneous capture and release of payloads, depending on the surrounding conditions. It is further noted that there is no requirement for the compartment to fuse with cells in order to deliver a payload, as is the case, e.g., for liposomes. A further advantage of the permeability and “spontaneity” of the compartments is that it may capture or release a payload in response to a change in environmental conditions, or stimuli. The compartments of the invention may be used, for example, for delivery of molecules to the extracellular matrix (possibly for enzyme replacement), for local (topical) delivery of active agents, or for capturing access molecules.

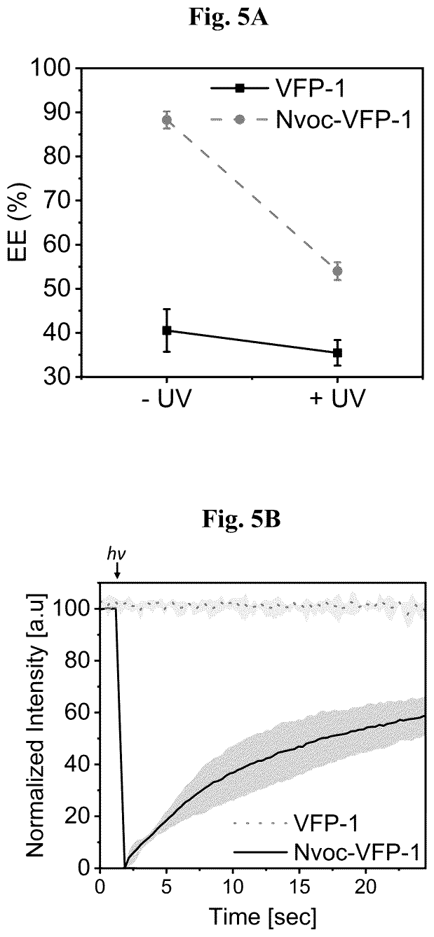

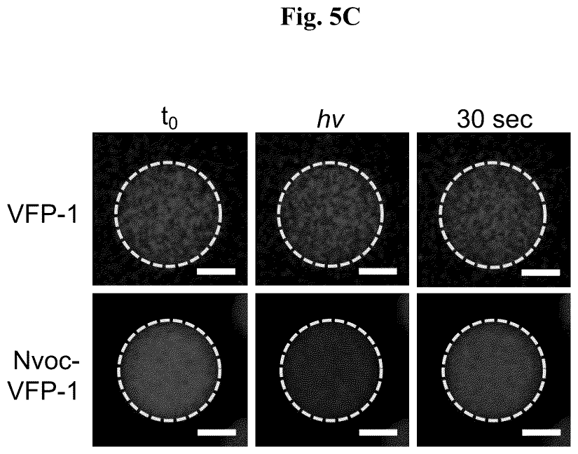

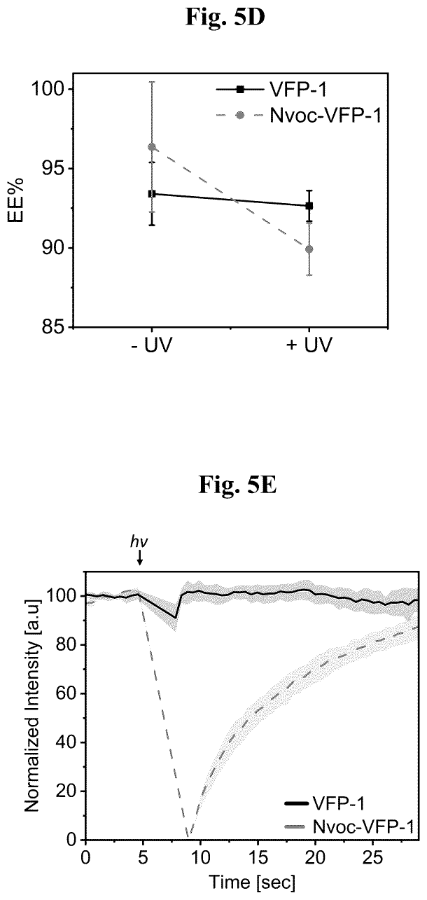

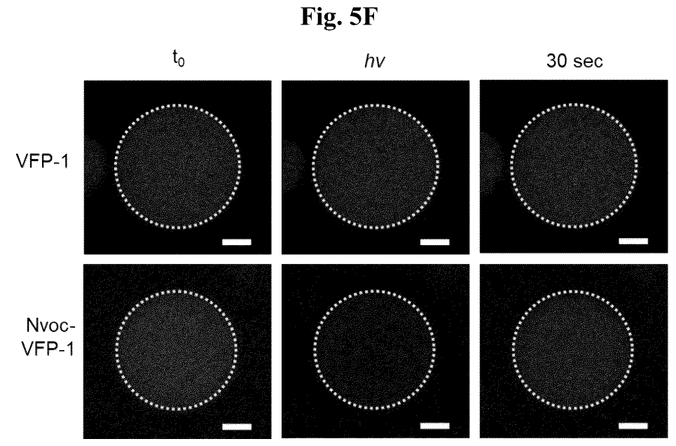

Being a micron-size compartment, it is capable of encapsulating macromolecules and even particles. Further, the compartments of the invention are also able to encapsulate molecules regardless of charge, such nucleic acids and charged molecules, including RNA. Finally, encapsulation by the compartment of the invention is high, and may reach above 90% (see FIGS. 5A and 5D).

The present invention is directed to LLPS-based compartments that can efficiently encapsulate and release payloads. In order to prepare a simple and controllable system, the compartments of the present application are based on a combination of a short peptide and anionic polymer such as RNA.

Peptides are attractive building blocks for construction of functional biomaterials including those composed of liquid assemblies, as their side-chain groups provide a diverse set of simple chemical functionalities that collectively constitute a rich and versatile chemical space. Unlike the case for proteins, the peptide composition, even at the single-amino acid level, directly dictates the supramolecular structure and material properties, thereby enabling sequence-structure and structure-function relationships to be established.

The compartments of the invention are based on viral factories formed by measles virus as a model system. The first peptide selected for the compartments of the invention, herein termed viral factory-inspired peptide (VFP-1), was defined based on searches for prevalent amino acids in the disordered phosphate-binding loop (P loop) of the phosphoprotein (P protein), one of the two main building blocks of measles viral factories. As shown, the designed VFP-1 peptide, which contains prevalent amino acids from the P protein P loop, efficiently forms liquid droplets (compartments) upon complexation with RNA.

The analyses presented in the experimental section of the present application demonstrate that the material properties of the compartments may be easily modulated by changes to the peptide sequence, at a single amino acid level. A further level of the compartments includes peptides modified by a light-cleavable group and demonstrates light-induced tunable dynamics, which shows the effect of charge and polarity of the peptide on the properties of the compartments. The present application further shows the effect of these changes on controlled partitioning and release of payloads from the compartments.

In some embodiments, the present invention provides a composition comprising a liquid-liquid phase separation (LLPS)-based compartment comprising a cationic peptide and an anionic polymer.

The term “compartment”, as used herein, relates to micron-scale, liquid-like, membraneless body or assembly, also referred to as a biomolecular condensate. Such compartments exists in mammalian cells and usually include proteins and nucleic acids. They are capable of compartmentalizing reactions such as viral replication and assembly of viral particles, and are therefore capable of trapping various molecules.

The term “liquid-liquid phase separation (LLPS)-based compartment” relates to compartments which are formed by the process of LLPS. LLPS is a thermodynamically-driven, reversible phenomenon consisting in de-mixing into two distinct liquid phases, with different solute concentrations. The equilibrium between mixing and de-mixing is strongly dependent on the chemical composition of the components and their concentrations, temperature, pressure, pH, ionic strength, crowding agents, etc.

In some embodiments, the present invention provides an LLPS-based compartment comprising a cationic peptide and an anionic polymer.

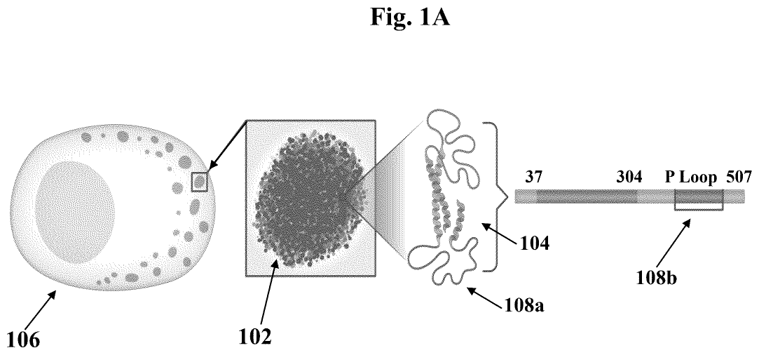

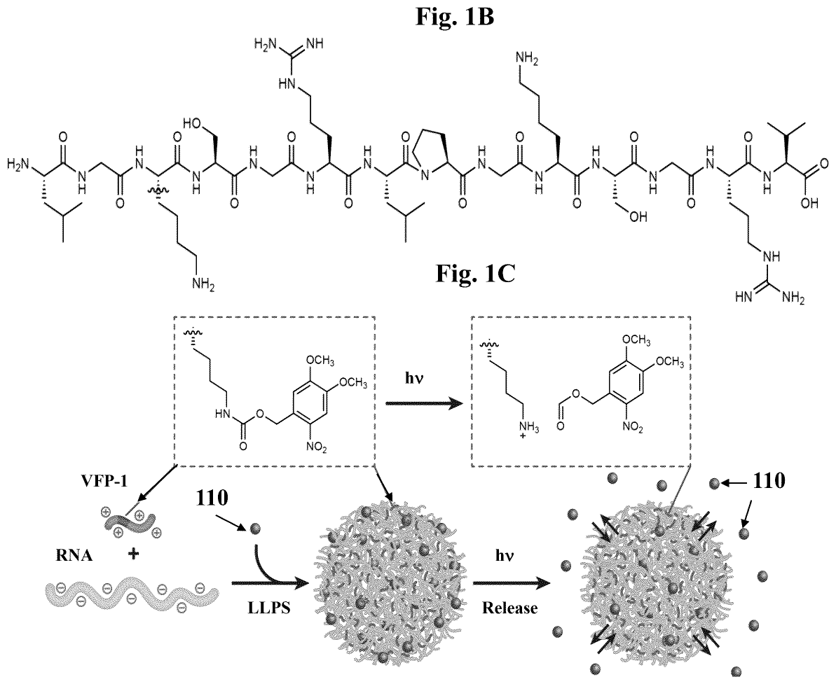

FIG. 1A is a schematic illustration showing a measles viral compartment 102 in the context of a mammalian cell 106, which has inspired the compartments of the present invention. The viral compartment 102 comprises a measles virus phosphoprotein (P protein) 104, which includes an unordered P Loop region 108a. A diagram of the P protein is presented on the right side of the figure, and shows the P Loop region 108b in the C-terminal side of the protein.

In some embodiments, the compartment has a size in the micron range. In some embodiments, the size of the compartment is about between about 100 nm to about 10 micron. As a result of the large size of the compartment, it may not readily enter into cells. The compartment diameter may be regulated by varying the RNA concentration.

In some embodiments, a compartment diameter of about 100 nm-1000 nm is obtained by using an anionic polymer (such as RNA, e.g., poly-U RNA) concentration of between about 0.1-0.5 mg/ml. In some embodiments, an anionic polymer concentration of above 0.5 mg/ml leads to a compartment diameter higher than 1000 nm. In some embodiments, an anionic polymer concentration of below 0.1 mg/ml leads to a compartment diameter lower than 100 nm.

The compartment, or condensate, generally has a variable density, with an outer, more dilute, phase, and an inner, denser phase. The compartment is capable of trapping molecules, such as enzymes, proteins, small molecules, and nucleic acids, which may transition between the inner and the outer phase of the compartment by diffusion, and also between the compartment inside and outside.

Due to the large size of the compartment, it is capable of trapping rather large molecules, such as large hydrophobic molecules which are usually difficult to solubilize.

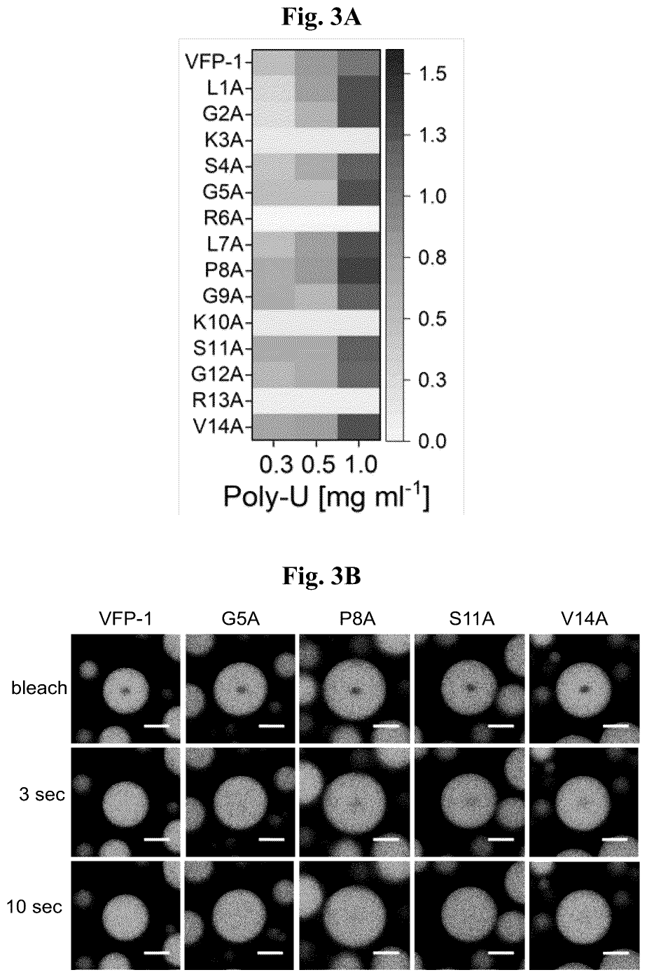

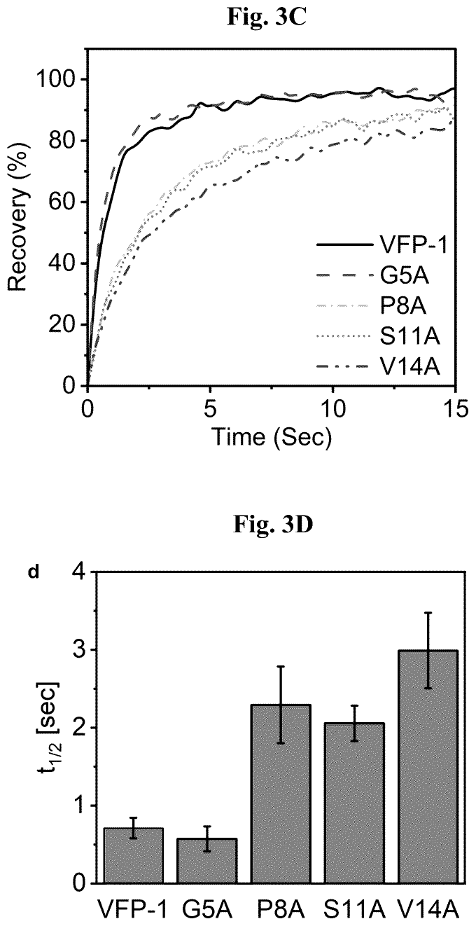

As shown in FIG. 3B-3D, the compartment of the invention shows a fast recovery following photobleaching. Accordingly, in some embodiments, the compartment has a recovery t½ after photobleaching of less than about 10, 8, 5, 3, 2, 1, or 0.5 sec. Accordingly, in some embodiments, the compartment has a diffusion rate of at least about 1, 2, 5, 8, 10, or 15×10⁻⁸ m²/sec.

In some embodiments, at least about 60, 70, 80, or 90% of the compartment recovers within about 10 seconds after photobleaching. In some embodiments, at least about 60, 70, 80, or 90% of the compartment recovers within about 15 seconds after photobleaching.

FIGS. 5A and 5B show encapsulation efficiency of the compartment of the invention.

In some embodiments, the encapsulation efficiency of the compartment of the invention for a payload is at least about 60, 70, 80, or 90%.

The compartments of the invention were inspired by viral compartment formed by the measles virus phosphoprotein (P protein) (FIG. 1A) and other viral proteins. However, large proteins, such as those present in natural viral compartment, are complex and difficult to work with. It is further difficult to control their properties, including entrapment and release of a payload. Accordingly, the 14-mer viral factory-inspired peptide (VFP-1) LGKSGRLPGKSGRV (SEQ ID NO: 1, shown in FIG. 1B) used as a basis for the experiments presented herein was designed based on prevalence of amino acids in the phosphate-binding loop (P-loop) of the P protein.

The advantage of using a short peptide is that it is easy to control the compartment properties by modifying even a single amino acid, and further control entrapment and release of a payload.

In some embodiments, the cationic peptide has a length of about 5-20 amino acids. In some embodiments, the cationic peptide has a length of about 9-20 amino acids. In some embodiments, the cationic peptide has a length of about 9-15 amino acids. In some embodiments, the cationic peptide has a length of about 14 amino acids.

The composition of the sequence of the cationic peptide may affect the features of the LLPS-based compartments, as shown throughout the application.

Accordingly, in some embodiments, the peptide comprises an amino acid sequence of a protein phosphate-binding loop (P-loop). In some embodiments, the peptide comprises an amino acid sequence of a P loop of a viral phosphoprotein (P protein).

In some embodiments, the amino acids of the peptide of the invention appear in the peptide at about the same frequencies as they appear in the P loop. In some embodiments, the peptide comprises basic amino acids, hydrophobic amino acids, glycine, and optionally aromatic amino acids at a about the same frequencies as they appear in the P loop. In some embodiments, the frequencies of Arg+Lys, Leu+Val, Gly, and optionally Phe, are about the same as their frequencies in the P loop.

In some embodiments, the P protein is derived from a virus selected from a measles virus, a rabies virus, and a SARS-CoV-2 virus.

As shown in FIG. 3A, changing the basic (positively-charged) residues of the peptide (K3, R6, K10, R13) into Alanine completely abolished the ability of the peptide to form a compartment by LLPS.

Accordingly, in some embodiments, the cationic peptide comprises at least one positively-charged amino acid. In some embodiments, the cationic peptide comprises at least two positively-charged amino acid. In some embodiments, the cationic peptide comprises at least three positively-charged amino acid. In some embodiments, the cationic peptide comprises at least four positively-charged amino acid.

In some embodiments, at least 10, 15, 20, or 25% of the amino acids of the cationic peptide are positively-charged amino acid. In some embodiments, at least 10, 15, 20, or 25% of the amino acids of the cationic peptide are selected from arginine (Arg, R), lysine (Lys, K), and histidine (His, H). In some embodiments, at least 10, 15, 20, or 25% of the amino acids of the cationic peptide are selected from Arg and Lys. In some embodiments, about 10-30% of the amino acids of the cationic peptide are selected from Arg and Lys.

Additional amino acids are also expected to affect the properties of the compartment, as shown, e.g., in FIG. 3B-3D, which demonstrate the effect of various amino acid substitutions on the recovery rate after photobleaching (FRAP), corresponding to the diffusion rate of the compartment. This example shows that, the material properties of the compartments can be tuned by simple changes to the peptide sequence, using systematic alanine (Ala) scanning analysis and construction of 14 sequence variants. The results also demonstrate that while the main driving forces of LLPS and droplet formation are electrostatic interactions between the peptide and RNA, additional modes of interactions mediate LLPS, as specific Ala substitutions of non-basic amino acids affect the diffusivity of the compartments.

In general, glycine (Gly, G), which is a very small amino acid, may allow flexibility in the disordered peptide chain, which is key for LLPS, while the basic Arg and Lys would promote electrostatic or cation-π interaction with the anionic polymer (e.g., RNA). Non-polar (hydrophobic) amino acids such as valine (Val, V) and leucine (Leu, L) would promote hydrophobic interactions between the peptide molecules, and aromatic amino acids such as phenylalanine (Phe, F) may be useful for facilitating LLPS with certain anionic polymers.

In some embodiments, the peptide further comprises at least one hydrophobic amino acid. In some embodiments, the hydrophobic amino acid is selected from Leu, Val, Alanine (Ala, A), isoleucine (Ile, I), and methionine (Met, M). In some embodiments, at least 10, 15, or 20% of the amino acids of the cationic peptide are hydrophobic amino acid. In some embodiments, about 10-30% of the amino acids of the cationic peptide are hydrophobic amino acid. In some embodiments, at least 10, 15, or 20% of the amino acids of the cationic peptide are selected from Leu, Val, Ala, Ile, and Met. In some embodiments, at least 10, 15, or 20% of the amino acids of the cationic peptide are selected from Leu and Val. In some embodiments, about 10-30% of the amino acids of the cationic peptide are Leu or Val.

In some embodiments, the peptide further comprises at least one Gly, proline (Pro, P), Serine (Ser, S), and/or threonine (Thr, T). In some embodiments, at least 10, 15, or 20% of the amino acids of the cationic peptide are selected from Gly, Pro, Ser, and Thr. In some embodiments, at least 10, 15, or 20% of the amino acids of the cationic peptide are Gly. In some embodiments, about 10-30% of the amino acids of the cationic peptide are Gly.

In some embodiments, the peptide further comprises at least one aromatic amino acid. In some embodiments, the aromatic amino acid is selected from phenylalanine (Phe), tyrosine (Tyr), Histidine (His), and tryptophan (Trp).

In some embodiments, the peptide comprises at least one of Arg and Lys; and at least one of Leu, Val, Ala, Ile, and Met. In some embodiments, the peptide comprises at least one of Arg and Lys; and at least one of Leu and Val.

In some embodiments, the peptide comprises at least one of Arg and Lys; and at least one of Phe, Tyr, His, and Trp.

In some embodiments, the peptide comprises at least one of Arg and Lys; at least one of Leu, Val, Ala, Ile, and Met; and at least one of Phe, Tyr, His, and Trp. In some embodiments, the peptide comprises at least one of Arg and Lys; at least one of Leu and Val; and at least one of Phe, Tyr, His, and Trp.

In some embodiments, the peptide comprises at least one of Arg and Lys; at least one of Leu, Val, Ala, Ile, and Met; and at least one of Gly, Pro, Ser, and Thr. In some embodiments, the peptide comprises at least one of Arg and Lys; at least one of Leu and Val; and at least one of Gly, Pro, Ser, and Thr. In some embodiments, the peptide comprises at least one of Arg and Lys; at least one of Leu, Val, Ala, Ile, and Met; and at least one Gly. In some embodiments, the peptide comprises at least one of Arg and Lys; at least one of Leu and Val; and at least one Gly.

In some embodiments, the peptide comprises at least one of Arg and Lys; at least one of Leu, Val, Ala, Ile, and Met; at least one of Phe, Tyr, His, and Trp; and at least one of Gly, Pro, Ser, and Thr. In some embodiments, the peptide comprises at least one of Arg and Lys; at least one of Len and Val; at least one of Phe, Tyr, His, and Trp; and at least one of Gly, Pro, Ser, and Thr. In some embodiments, the peptide comprises at least one of Arg and Lys; at least one of Leu, Val, Ala, Ile, and Met; at least one of Phe, Tyr, His, and Trp; and at least one Gly. In some embodiments, the peptide comprises at least one of Arg and Lys; at least one of Len and Val; at least one of Phe, Tyr, His, and Trp; and at least one Gly.

In some embodiments, the peptide comprises at least one of Arg and Lys; at least one of Leu, Val, Ala, Ile, and Met; at least one Phe; and at least one of Gly, Pro, Ser, and Thr. In some embodiments, the peptide comprises at least one of Arg and Lys; at least one of Len and Val; at least one Phe; and at least one of Gly, Pro, Ser, and Thr. In some embodiments, the peptide comprises at least one of Arg and Lys; at least one of Leu, Val, Ala, Ile, and Met; at least one Phe; and at least one Gly. In some embodiments, the peptide comprises at least one of Arg and Lys; at least one of Len and Val; at least one Phe; and at least one Gly.

In some embodiments, the peptide comprises at least 10% of each of: positively-charged amino acids, hydrophobic amino acids, and Gly. In some embodiments, the peptide comprises at least 20% of each of: positively-charged amino acids, hydrophobic amino acids, and Gly. In some embodiments, the peptide comprises about 10-30% of each of: positively-charged amino acids, hydrophobic amino acids, and Gly. In some embodiments, the peptide comprises about 10-30% of each of: Arg and/or Lys, Val and/or Leu, and Gly. In some embodiments, the peptide further comprises at least one Phe. In some embodiments, the peptide further comprises at least one Pro.

In some embodiments, the peptide does not comprise an acidic (anionic) amino acid, such as aspartic acid (Asp, D) or glutamic acid (Glu, E).

In some embodiments, the positively-charged amino acids in the peptide are not adjacent to one another. In other words, no two positively-charged amino acids (such as arg, lys, or His) are in adjacent positions. In some embodiments, every two positively-charged amino acids are separated by at least one non-positively-charged amino acid. In some embodiments, the positively-charged amino acids are generally uniformly distributed throughout the peptide sequence. For example, each two positively-charged amino acids are separated by about the same number of non-positively-charged amino acids (plus or minus one amino acid). In some embodiments, the positively-charged amino acids are separated by 1-3 non-positively-charged amino acids.

In some embodiments, at least one pair of positively-charged amino acids are not separated by a non-positively-charged amino acid.

In some embodiments, the non-positively-charged amino acids are selected from Gly, Pro, Ser, Thr, Leu, Val, Ala, Ile, Met, Phe, Tyr, and Trp.

In some embodiments, the peptide comprises a sequence selected from the sequences SEQ ID NOs: 1, 2, 3, 5, 6, 8, 9, 10, 12, 13, and 15.

In some embodiments, the peptide sequence is selected from the sequences SEQ ID NOs: 1, 2, 3, 5, 6, 8, 9, 10, 12, 13, and 15.

In some embodiments, the peptide comprises a sequence selected from the sequences SEQ ID NOs: 1, 6, 8, 9, 12, and 15.

In some embodiments, the peptide sequence is selected from the sequences SEQ ID NOs: 1, 6, 8, 9, 12, and 15.

In some embodiments, the peptide comprises a sequence as set forth in SEQ ID NOs: 1.

In some embodiments, the peptide sequence is as set forth in SEQ ID NOs: 1.

In some embodiments, the peptide includes at least one modified amino acid. In some embodiments, the modified amino acid is a positively-charged amino acid. In some embodiments, the modification abolishes the positive charge of the amino acid that was modified. In some embodiments, the modified amino acid is modified by the addition of an aromatic or a hydrophobic group. In some embodiments, the peptide includes more than one modified amino acids.

In some embodiments, the cationic peptide is one or more peptide selected from the peptides mentioned above. In some embodiments, the cationic peptide is a combination or a fusion of more than one different cationic peptides.

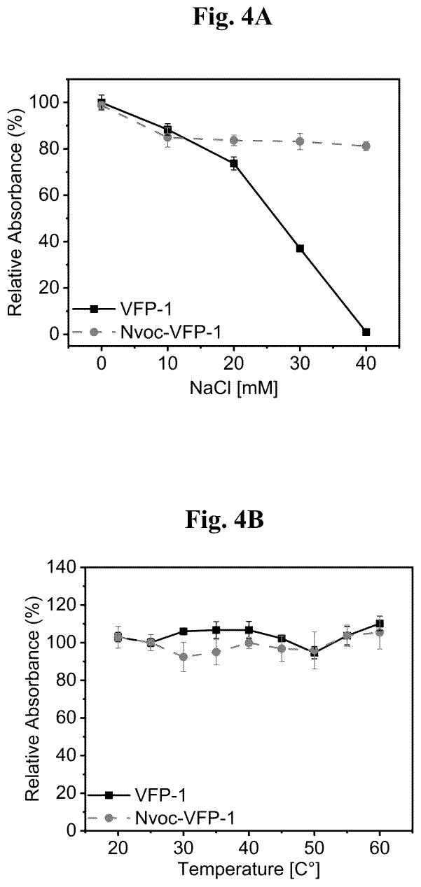

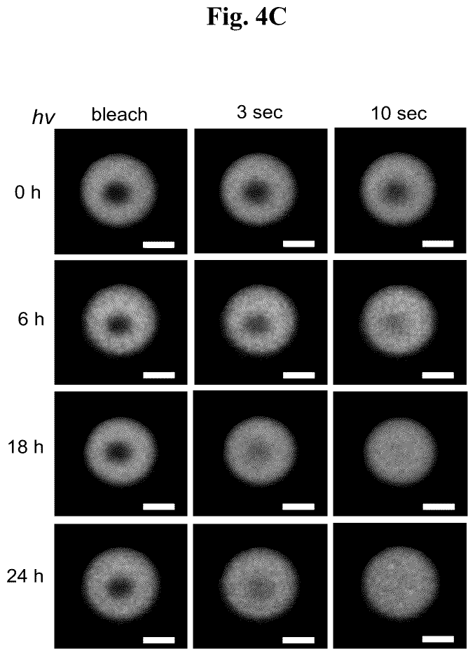

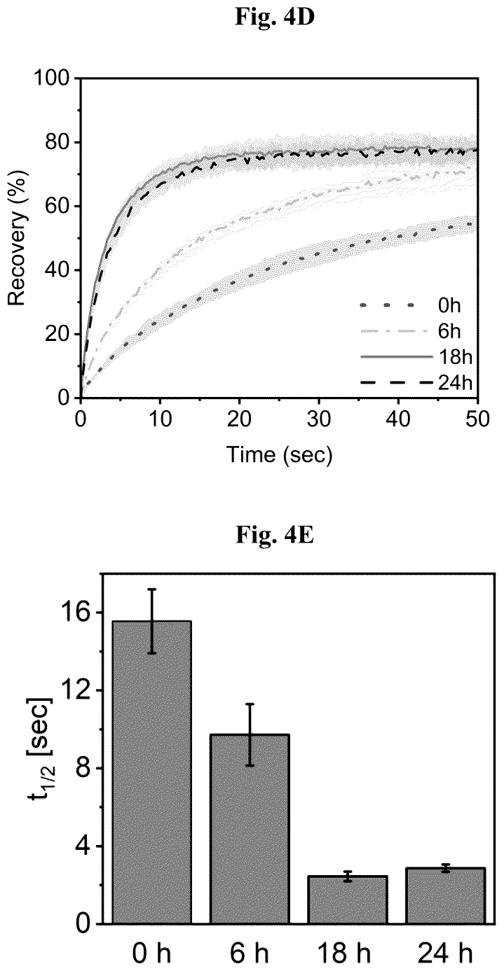

As shown in Example 4, modification of one of the positively-charged amino acids of the peptide, in this case Lys at position 3, changed the material properties of the peptide. The modifying group used was o-nitroveratryloxycarbonyl (Nvoc) (FIG. 1C, the resulting peptide having the sequence set forth in SEQ ID NO: 16), which is a hydrophobic aromatic group, cleavable by irradiation. This modification reduced the positive charge and polarity of the peptide, and as shown in FIG. 4, also affected its properties. A can be seen from FIGS. 4C-4E, this modification decreased droplet dynamics as shown by the increased t½ for recovery after photobleaching, probably caused by promoting hydrophobic or π-π interactions between the peptide molecules or between the peptide and the RNA. Photocleavage of the Nvoc increased the droplet dynamics, probably by increasing peptide polarity, reducing peptide-peptide contacts, and promoting electrostatic interactions between the peptide and RNA.

This exemplifies the ability to quickly and easily control the properties of the compartment. Such changing of properties may be used, e.g., for controlling entrapment and release of a payload.

Accordingly, in some embodiments, the peptide comprises at least one modified amino acid. In some embodiments, the modified amino acid is modified by a cleavable group. In some embodiments, the cleavable group is a protecting group. In some embodiments, the peptide is modified by at least one cleavable group which is bound to at least one amino acid.

In some embodiments, the cleavable group is a hydrophobic, or a non-polar, cleavable group. In some embodiments, the cleavable group is an aromatic cleavable group.

In some embodiments, the cleavable group is cleavable by light (photocleavable). In some embodiments, the cleavable group is cleavable by an enzymatic reaction (enzymatically cleavable). In some embodiments, the cleavable group is cleavable by a chemical reagent or by applying certain conditions, such as pH, temperature, ionic strength, etc.

Photocleavable groups are cleavable by irradiation with light at a certain wavelength range. Nonlimiting examples for photocleavable groups suitable for use with the invention include Nitrobenzyl-based groups such ortho-nitrobenzyl (o-nitrobenzyl, ONB), and o-nitroveratryloxycarbonyl (Nvoc); and BODIPY (C₉H₇BN₂F₂ or 4,4-difluoro-4-bora-3a,4a-diaza-s-indacene)-derived photocleavable protecting groups.

In some embodiments, the light used for cleaving the photocleavable group is in the blue light region. In some embodiments, the light used for cleaving the photocleavable group is in the UV light region. In some embodiments, the light used for cleaving the photocleavable group is at a wavelength of above 300, 400, 500, or 600 nm. In some embodiments, the light is at a wavelength of about 300-800, 300-700, 300-600, 300-500, 300-450, 350-800, 350-700, 350-500, 350-450, 380-700, 380-600, 380-500, 400-700, 400-600, 400-500, or 450-500 nm. In some embodiments, the light is at a wavelength of about 300 to about 450 nm. In some embodiments, the light is at a wavelength of about 365 nm or about 405 nm. In some embodiments, the light is at a wavelength of about 365 nm.

Nonlimiting examples for enzymatically cleavable groups suitable for use with the invention include tert-butyl cleavable groups such as BOC (tert-butyloxycarbonyl, or di-tert-butyl dicarbonate (Boc2O)), Fluorenylmethyloxycarbonyl chloride (Fmoc-Cl), which may be removed by an esterase from Bacillus subtilis (BsubpNBE) or lipase A from Candida antarctica (CAL-A), or alternatively by the addition of a certain reagent or under specific conditions.

Additional nonlimiting examples for suitable cleavable groups include carbamate cleavable groups such as pyridoxal 5′-phosphate (PLP) cleavable aminobutanamide carbamate (abac), periodate cleavable aminobutanol carbamate (aboc), and aryldithioethyloxycarbonyl (Ardec) removable under mild reducing conditions.

In some embodiments, the peptide is modified on a polar amino acid. In some embodiments, the peptide is modified on a positively-charged (or basic) amino acid. In some embodiments, the peptide is modified on a Lys or an Arg. In some embodiments, the peptide is modified on a Lys.

In some embodiments, the peptide is modified on more than one amino acid. In some embodiments, the peptide is modified on two amino acids. In some embodiments, the peptide is modified on three amino acids. In some embodiments, the peptide is modified by one cleavable group, such as that if the peptide is modified on multiple amino acids, then the same cleavable group is bound to each of the modified amino acids. In some embodiments, the peptide is modified by different cleavable group. In some embodiments, the peptide is modified by different cleavable groups, the different cleavable groups being cleavable under different conditions. For example, the peptide may be modified by a photocleavable group and an enzymatically cleavable group, or by two photocleavable groups which are cleavable at different wavelengths.

In some embodiments, the peptide comprises a sequence as set forth in SEQ ID NO: 16, including a modification of Lys at position 3 of SEQ ID NO: 1 by Nvoc. In some embodiments, the peptide has a sequence as set forth in SEQ ID NO: 16

In some embodiments, the cleavable group is one or more cleavable group selected from the cleavable groups mentioned above.

A variety of anionic polymers may be suitable for use with the present invention, including natural and synthetic anionic polymers or combinations thereof. Nonlimiting examples of anionic polymers suitable for use with the invention include negatively charged nucleic acids, such as RNA, ssDNA, dsDNA, and non-RNA non-DNA or modified nucleic acids; and non-nucleic acid negatively charged polymers such as heparin and hyaluronic acid, or other modified polymers, e.g., phosphorylated polymers. The negatively-charged anionic polymers, such as the RNA, may have a specific structure such as a hairpin loop (structured, or ordered), or lacking a specific structure (unstructured, or unordered), and combinations thereof, i.e., polymers having both structured and unstructured regions. Some specific examples include a poly uracil (poly-U) RNA, siRNA, snRNA, shRNA, microRNA, tRNA, and combinations thereof.

In some embodiments, the anionic polymer is a natural anionic polymer. In some embodiments, the anionic polymer is a synthetic anionic polymer. In some embodiments, the anionic polymer is a combination of a natural anionic polymer and a synthetic anionic polymer.

In some embodiments, the anionic polymer is selected from heparin, hyaluronic acid, and a nucleic acid. In some embodiments, the anionic polymer is a nucleic acid. In some embodiments, the anionic polymer is RNA. In some embodiments, the anionic polymer is an oligonucleotide. In some embodiments, the anionic polymer is a polynucleotide. In some embodiments, the anionic polymer is a poly-U RNA. In some embodiments, the anionic polymer is an oligo-dT ssDNA.

In some embodiments, the anionic polymer is a combination of different anionic polymers. In some embodiments, the anionic polymer is one or more of the anionic polymers mentioned above.

The examples show successful LLPS for the VFP-1 peptide of the invention and its derivatives with anionic polymers of various sizes, from about 70 nucleotides of single strand (ss)DNA (approximately 20-30 kDa) to poly-U RNA having around 3000 bases (approximately 1000 kDa). It therefore appears that a wide range of sizes of the anionic polymer may be used in the compartments of the invention. Therefore, in some embodiments, the anionic polymer may be of any size. In some embodiments, the anionic polymer has a molecular weight of about 10-10000, 10-5000, 20-3000, 20-2000,20-1000, 20-500,20-300, 20-100, 50-2000, 50-1000, 50-500, 50-300, 50-100, 100-10000, 100-5000, 100-3000, 100-2000, 100-1000, 1000-10000, 1000-5000, or 1000-3000 kDa.

In some embodiments, the anionic polymer is unordered, i.e., does not form an ordered structure, and does not comprise a part which forms an ordered structure. In some embodiments, the anionic polymer comprises at least one ordered structure. In some embodiments, at least 20%, 30%, 40%, 50%, 70%, or 80% of the anionic polymer forms at least one ordered structure. In some embodiments, at least 20%, 30%, 40%, 50%, 70%, or 80% of the anionic polymer is unordered, i.e., does not form an ordered structure. In some embodiments, the complete anionic polymer is comprised in at least one ordered structure. In some embodiments, the complete anionic polymer comprises several ordered structures. In some embodiments, the anionic polymer does not comprise an unordered structure.

In some embodiments, the anionic polymer comprises at least one secondary structure. In some embodiments, the anionic polymer comprises at least one stem and loop structure. In some embodiments, the anionic polymer comprises more than one secondary, or stem and loop structures. In some embodiments, at least 70%, 80%, or 90% of the anionic polymer is comprised in stem and loop structures.

An “ordered structure”, as used herein, means a non-random structure, in which at least some intramolecular base pairing occurs which defines a secondary structure, such as a hairpin or a stem and loop structure. Accordingly, an ordered structure or region is intended to include a secondary structure, such as a stem and loop structure. A poly-U RNA or poly-dT DNA are examples for an unordered structure, since no base pairing can occur between the all-U or all-T bases. On the other hand a tRNA, having several stem and loop structures is an example for a nucleic acid comprising ordered structures.

In some embodiments, the anionic polymer comprises at least one structured region (e.g., a secondary structure such as a stem and loop region) and the cationic peptide comprises at least one aromatic or modified amino acid (e.g., an amino acid modified by a cleavable group such as Nvoc).

In some embodiments, at least 20%, 30%, 40%, 50%, 70%, or 80% of the anionic polymer comprises at least one ordered structure, and the cationic peptide comprises at least one aromatic or modified amino acid.

In some embodiments, the complete anionic polymer is comprised in at least one ordered structure, and the cationic peptide comprises at least one aromatic or modified amino acid.

In some embodiments, the complete anionic polymer does not comprise an unordered structure, and the cationic peptide comprises at least one aromatic or modified amino acid.

In some embodiments, at least 20%, 30%, 40%, 50%, 70%, or 80% of the anionic polymer is unordered, and the cationic peptide comprises at least one aromatic or modified amino acid.

In some embodiments, the anionic polymer is unordered, and the cationic peptide comprises at least one aromatic or modified amino acid.

In some embodiments, the anionic polymer comprises at least one structured region and the cationic peptide does not comprise an aromatic or modified amino acid.

In some embodiments, at least 20%, 30%, 40%, 50%, 70%, or 80% of the anionic polymer forms at least one ordered structure, and the cationic peptide does not comprise an aromatic or modified amino acid.

In some embodiments, the complete anionic polymer is comprised in at least one ordered structure, and the cationic peptide does not comprise an aromatic or modified amino acid.

In some embodiments, the anionic polymer does not comprise an unordered structure, and the cationic peptide does not comprise an aromatic or modified amino acid.

In some embodiments, at least 20%, 30%, 40%, 50%, 70%, or 80% of the anionic polymer is unordered, and the cationic peptide does not comprise an aromatic or modified amino acid.

In some embodiments, the anionic polymer is unordered, and the cationic peptide does not comprise an aromatic or modified amino acid.

In some embodiments, the at least one aromatic amino acid is selected from Phe, Tyr, His, and Trp. In some embodiments, the at least one aromatic amino acid is Phe. In some embodiments, the modified amino acid is modified by an aromatic and/or hydrophobic group. In some embodiments, the modified amino acid is modified by an aromatic and/or hydrophobic cleavable group. In some embodiments, the modified amino acid is modified by Nvoc.

In some embodiments, the anionic polymer is a nucleic acid. In some embodiments, the anionic polymer is RNA. In some embodiments, the anionic polymer comprises at least one secondary structure. In some embodiments, the anionic polymer comprises at least one stem and loop structure. In some embodiments, the anionic polymer comprises more than one stem and loop structure. In some embodiments, at least 70%, 80%, or 90% of the anionic polymer comprises stem and loop structures. In some embodiments, the anionic polymer is tRNA or has a tRNA-like structure.

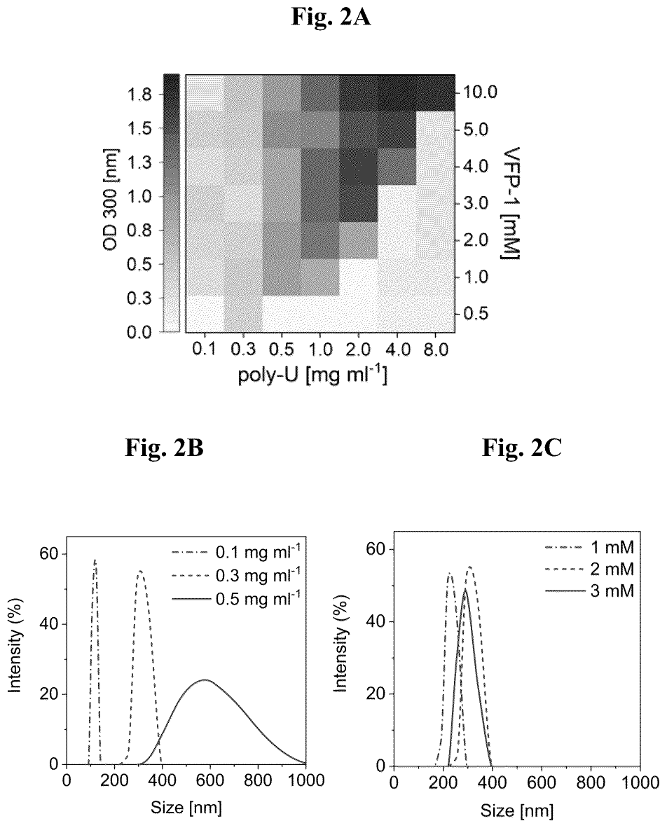

FIG. 2A shows LLPS between increasing concentrations of the VFP-1 peptide and poly-U RNA, where some LLPS is already detectable at low concentrations such as 0.5 mM of peptide and 0.1 mg/ml of RNA. The absorbance increases as both components increase in concentration.

Based on that at pH=7.5 the peptide has 4 positive charges per molecule and the poly-U RNA has about 2940 negative charges per molecule, a ratio of about 2.4 positive to negative charges was calculated as the minimal ratio for LLPS.

Accordingly, in some embodiments, the positive charges from the cationic peptide and the negative charges from the anionic polymer are at a ratio of at least about 2:1 positive to negative charges in the composition.

In some embodiments, the ratio of positive charges to negative charges in the composition is about 2:1 to about 10:1. In some embodiments, the ratio of positive charges to negative charges in the composition is at least about 2:1, 2.5:1, 3:1, 3.5:1, 4:1, 4.5:1, or 5:1. In some embodiments, the ratio of positive charges to negative charges in the composition is about 2:1, 2.5:1, 3:1, 3.5:1, 4:1, 4.5:1, or 5:1. In some embodiments, the ratio of positive charges to negative charges in the composition is at least about 2.4.

In some embodiments, the pH of the composition is about 7 to about 8. In some embodiments, the pH of the composition is about 7.5.

In some embodiments, the concentration of the cationic peptide in the composition is at least about 0.5, 1, 1.5, 2, 2.5, 3, 3.5, 4, 4.5, or 5 mM. In some embodiments, the concentration of the cationic peptide in the composition is at least about 2, 3, or 5, mM. In some embodiments, the concentration of the cationic peptide in the composition is about 0.5-10, 1-10, 1-5, 1-3, 2-10, 2-5, 3-10, 3-5, or 5-10, mM.

In some embodiments, the concentration of the anionic polymer in the composition is at least about 0.1 or 0.3 mg/ml. In some embodiments, the concentration of the anionic polymer in the composition is about 0.1-8, 0.1-1, 0.1-0.5, or 0.1-0.3, mg/ml. In some embodiments, the concentration of the anionic polymer in the composition is at least about 0.1, 0.3, 0.5, or 1, mg/ml.

In some embodiments, the concentration of the cationic peptide in the composition is about 1-3 mM and the concentration of the anionic polymer in the composition is about 0.1-0.3 mg/ml.

As shown in FIG. 4A, the Nvoc-modified peptide was not sensitive to increasing salt concentrations probably due to π-π interactions formed by the Nvoc group, while the unmodified peptide started disintegrating at NaCl concentrations of about 10 mM, and almost completely disintegrated at about 40 mM NaCl. This feature may be used for releasing a payload from the compartment either by increasing the salt concentration when the peptide is not modified (or does not comprise an aromatic amino acid that may have a similar effect), or when the peptide is modified by a cleavable aromatic group, cleaving the aromatic group at a high salt concentration to release the payload.

Accordingly, in some embodiments, the composition further comprises salt. In some embodiments, the composition comprises at least about 10, 20, 30, or 40, mM salt. In some embodiments, the salt is a monovalent salt. In some embodiments, the salt is NaCl.

In some embodiments, the composition further comprises salt and the cationic peptide comprises an amino acid modified by a cleavable aromatic group. In some embodiments, the composition further comprises at least about 10, 20, 30, or 40, mM salt and the cationic peptide comprises an amino acid modified by a cleavable aromatic group. In some embodiments, the composition further comprises a payload.

In some embodiments, the composition is a pharmaceutical composition, further comprising a pharmaceutically acceptable carrier.

Pharmaceutical compositions for use in accordance with the present invention may be formulated in any conventional manner using one or more physiologically or pharmaceutically acceptable carriers or excipients. The carrier(s) must be “acceptable” in the sense of being compatible with the other ingredients of the composition, not being deleterious to the recipient thereof, and not significantly interfering with the activity of the compartment of the invention or any payload comprised therein, or of any other active ingredient in the pharmaceutical composition. The term “pharmaceutically acceptable carrier” refers to a diluent, adjuvant, excipient, or vehicle with which the composition of the invention is administered. The carriers in the pharmaceutical composition may comprise a binder, such as microcrystalline cellulose, polyvinylpyrrolidone (polyvidone or povidone), gum tragacanth, gelatin, starch, lactose or lactose monohydrate; a disintegrating agent, such as alginic acid, maize starch and the like; a lubricant or surfactant, such as magnesium stearate, or sodium lauryl sulphate; and a glidant, such as colloidal silicon dioxide.

One of the uses of the compositions of the present invention, is encapsulation of payloads, e.g., for delivery to cells.

A variety of payloads may be suitable for encapsulation by the compartment of the invention. Nonlimiting examples for suitable payloads include proteins, nucleic acids, and small molecule drugs. Suitable proteins may include enzymes and antibodies, and suitable nucleic acids may include single-stranded or double-stranded DNA and RNA, for example siRNA, snRNA, shRNA, microRNA, etc.

In some embodiments, the composition further comprises at least one payload. In some embodiments, most of the payload molecules in the composition are entrapped inside the compartment. In some embodiments, at least 40, 50, 60, 70, 80, 90, 95, or 97% of the payload molecules in the composition are entrapped inside the compartment.

In some embodiments, the payload is hydrophobic, or non-polar. In some embodiments, the payload is hydrophilic, or polar. In some embodiments, the payload is positively-charged. In some embodiments, the payload is negatively-charged.

In some embodiments, the payload is a nucleic acid. In some embodiments, the payload is an RNA. In some embodiments, the payload is an DNA. In some embodiments, the payload is a protein. In some embodiments, the payload is an enzyme. In some embodiments, the payload is a small molecule, such as a small molecule drug.

In some embodiments, the size of the payload is about 100-100000 Da. In some embodiments, the size of the payload is about 100-50000, 100-20000, 100-10000, 100-5000 100-1000, 500-100000, 500-50000, 500-20000, 500-10000, 500-5000 500-1000, 1000-100000 1000-50000, 1000-20000, 1000-10000, 5000-100000 5000-50000, 5000-20000, or 5000-10000 Da.

In some embodiments, the size of the payload is about 100-1000 Da. In some embodiments, the size of the payload is about 200-1000, 500-1000, 100-800, 200-800, or 500-800 Da. In some embodiments, the payload has a size of about 500-800 Da. In some embodiments, the payload is hydrophobic and has a size of about 500-800 Da.

The dynamics of entrapment and release of a payload from the compartments of the invention may be controlled by various factors.

For example, when the cationic peptide is modified, e.g., by an aromatic or a hydrophobic group, release of the payload may be achieved by cleavage of the group by a suitable treatment, such as by irradiation, enzymatic, or chemical treatment of the compartment.

FIG. 1C shows an exemplified embodiment, where in the first step, the compartment is formed from a VFP-1 peptide modified by Nvoc on a lysine residue, RNA, and payload 110.

Following LLPS, the compartment is formed, encapsulating the payload. After irradiation at a wavelength which cases release of the Nvoc, the peptide becomes more polar and the payload is released.

FIGS. 5A and 5D demonstrate this experiment. As shown in FIG. 5A, rhodamine B (RhB) is initially about 90% encapsulated in the compartment comprising the Nvoc-VPF-1 peptide (and poly-U RNA). After irradiating, the Nvoc is released, and the encapsulation % us reduced by about 40%.

Accordingly, in some embodiments, the compartment is suitable for releasing the payload following treatment with a light trigger, a chemical agent, or an enzymatic trigger. In some embodiments, the cationic peptide is modified by a cleavable aromatic group, as defined above with respect to the cleavable groups.

Additionally, when the peptide is not modified (and/or does not comprise an aromatic amino acid), release of the payload may be achieved by the addition of salt to a concentration of 10-40 mM.

Accordingly, in some embodiments, the compartment is suitable for releasing the payload by elevating salt concentrations. In some embodiments, the cationic peptide is not modified by an aromatic group and does not comprise an aromatic amino acid.

The features of the payload may also affect the dynamics of entrapment and release. For example, as explained in Example 5, when the payload is structured (such as having a hairpin loop, as in a tRNA), and the peptide does not comprise an aromatic group, or an amino acid modified by an aromatic group, encapsulation occurred only at a high temperature (about 40-60° C.), and therefore release may be achieved by decreasing the temperature. On the other hand, when the payload is structured (such as having a hairpin loop, as in a tRNA), and the peptide comprises an aromatic group or an amino acid modified by an aromatic group (such as Nvoc), encapsulation occurs at lower temperatures, such as room temperature (about 25° C.).

Accordingly, in some embodiments, the compartment is suitable for releasing the payload by reducing the temperature. In some embodiments, the cationic peptide is not modified by an aromatic group and does not comprise an aromatic amino acid and the payload has at least one structured region, such as a hairpin loop. In some embodiments, the cationic peptide is modified by an aromatic group and/or comprises an aromatic amino acid, and the payload has at least one structured region, such as a hairpin loop.

Additionally, the features of the compartment change with varying pH, as the ratio of positive to negative charges changes. Accordingly, in some embodiments, the compartment is suitable for releasing the payload by changing the pH of the composition.

In some embodiments, there is provided the composition of the invention for use in the delivery of a payload to a subject.

In some embodiments, the payload is a therapeutic drug and the delivery is for treating a disease or condition in a subject in need thereof.

In some embodiments, the delivery is to an intracellular compartment, such as the extracellular matrix.

In some embodiments, there is provided the composition of the invention for use in the delivery of a payload to a cell.

In some embodiments, the cell is a procaryotic cell. In some embodiments, the cell is a eucaryotic cell. In some embodiments, the cell is a mammalian cell. In some embodiments, the cell is a cultured cell. In some embodiments, the cell is part of an organism. In some embodiments, the organism is a mammal. In some embodiments, the payload is a nucleic acid, such as DNA or RNA, and the cell is a mammalian cell.

The spontaneous response of the compartment of the invention to stimuli, such as light stimuli, also allows delivery of a payload to a specific site, by applying the stimulus at the specific site.

For example, the compartment of the invention may be used to deliver a drug to a tumor site, by forming a compartment loaded with the drug, wherein the cationic peptide comprises at least one amino acid modified by a photocleavable group which is cleavable at a certain wavelength or wavelength range. The compartment is designed such that following cleavage, the compartment will disassemble (as explained above, either based on conditions in the tumor environment, such as for example acidic pH, or by design of the anionic polymer structure). The loaded compartment is administering to a subject having a tumor, and light at the certain wavelength or wavelength range is applied to the tumor area, to cleave the payload and release the drug.

In some embodiments, there is provided a method for delivery of a drug to a tumor site, comprising forming the composition of the invention comprising a therapeutically effective amount of the drug and optionally a pharmaceutically acceptable carrier, wherein the cationic peptide comprises at least one amino acid modified by a photocleavable group which is cleavable at a certain wavelength or wavelength range, administering the composition to the subject, and applying to the tumor area light at the certain wavelength or wavelength range, thereby causing release of the drug at the tumor site.

In some embodiments, the compartment is designed such that when not including the photocleavable group (e.g., following cleavage) it disintegrates under conditions of the tumor microenvironment (e.g., a certain pH or salt ions, as explained above). In some embodiments, the compartment is designed such that when not including the photocleavable group (e.g., following cleavage) it disintegrates due to the anionic polymer structure, as explained above.

In some embodiments, the compartment comprises RNA as the anionic polymer, wherein the RNA comprises at least one secondary structure, such as a stem and loop structure.

The term “therapeutically effective amount” as used herein means an amount of the drug that will elicit the biological or medical response of a tissue, system, animal or human that is being sought, i.e. the therapeutic effect. The amount must be effective to achieve the desired therapeutic effect as described above, depending inter alia on the type and severity of the condition to be treated and the treatment regime. The therapeutically effective amount is typically determined in appropriately designed clinical trials (dose range studies) and the person skilled in the art will know how to properly conduct such trials to determine the effective amount. As generally known, an effective amount depends on a variety of factors including, the distribution profile within the body, a variety of pharmacological parameters such as half-life in the body, on undesired side effects, if any, and on factors such as age and gender, etc.

In some embodiments, the compartment of the invention is prepared by mixing the cationic peptide of the invention and the anionic polymer of the invention to allow forming a compartment by LLPS.

In some embodiments, the method further comprises adding a payload during the mixing. In some embodiments, the method further comprises adding a payload after the mixing, when the compartment has already been formed.

In some embodiments, the method further comprises adding salt during the mixing. In some embodiments, the salt is added to a concentration of at least about 10 mM. In some embodiments, the salt is added to a concentration of about 10, 15, 20, 25, 30, 35, 40, 45, or 50 mM. In some embodiments, the salt is a monovalent salt. In some embodiments, the salt is NaCl. In some embodiments, the cationic peptide comprises an aromatic amino acid or an amino acid modified by an aromatic group. In some embodiments, the cationic peptide comprises an amino acid modified by a cleavable aromatic group.

In some embodiments, the method further comprises elevating the temperature during the mixing. In some embodiments, the mixing is conducted at room temperature, such as at 25° C. In some embodiments, the mixing is conducted at a temperature of at least about 40, 50, or 60° C. In some embodiments, the mixing is conducted at a temperature of about 25, 30, 35, 40, 45, 50, 55, 60, 65, 70, 75, or 80° C. In some embodiments, the cationic peptide does not comprise an aromatic amino acid or an amino acid modified by an aromatic group. In some embodiments, the anionic polymer is ordered. In some embodiments, the anionic polymer comprises at least one ordered region. In some embodiments, the anionic polymer is a structured RNA, such as a tRNA.

Due to the open, membraneless structure of the compartment of the invention, it is capable of encapsulating and releasing a payload, or spontaneously forming and disintegrating, in response to change in environmental factors, or following a stimulus. Relevant changes in environmental factors may be, for example, changes in salt concentration, pH, temperature, and presence or absence of certain components. Further stimuli that may be applied to control the state of the compartment or release of payload, when the peptide comprises a cleavable group, include light, or the addition of an enzyme.

In some embodiments, there is provided a method for payload release from the compartment of the invention, comprising applying to the compartment light, an enzyme, or a reagent, wherein the peptide comprises a cleavable group, which is photocleavable, enzymatically cleavable, or cleavable by the reagent, as described above.

In some embodiments, the light is in the blue light region. In some embodiments, the light is in the UV light region. In some embodiments, the light is at a wavelength of above 300, 400, 500, or 600 nm. In some embodiments, the light is at a wavelength of about 300-800, 300-700, 300-500, 300-450, 350-800, 350-700, 350-500, 380-700, or 450-500 nm. In some embodiments, the light is at a wavelength of about 300 to about 450 nm. In some embodiments, the light is at a wavelength of about 365 nm or about 405 nm. In some embodiments, the light is at a wavelength of about 365 nm.

In some embodiments, the enzyme is an esterase or a lipase. In some embodiments, the enzyme is an esterase from Bacillus subtilis (BsubpNBE) or lipase A from Candida antarctica (CAL-A).

In some embodiments, there is provided a method for payload release from the compartment of the invention, comprising increasing the salt concentration. In some embodiments, the salt concentration is increased to above 10 mM. In some embodiments, the salt concentration is increased to above 10, 20, 30, 40 or 50 mM. In some embodiments, the salt concentration is increased to about 10, 20, 30, 40 or 50 mM. In some embodiments, the cationic peptide does not comprise an aromatic amino acid or an amino acid modified by an aromatic group.

In some embodiments, there is provided a method for payload release from the compartment of the invention, comprising reducing the temperature. In some embodiments, the temperature is reduced to 60, 50, 40° C. or below. In some embodiments, the temperature is reduced to room temperature, i.e., about 25° C. In some embodiments, the cationic peptide does not comprise an aromatic amino acid or an amino acid modified by an aromatic group. In some embodiments, the payload is ordered. In some embodiments, the payload comprises at least one ordered region. In some embodiments, the payload is a structured RNA, such as a tRNA.

In some embodiments, there is provided a method for payload release from the compartment of the invention, comprising changing the pH. This may be applicable to environments with a certain pH, which can cause release of the payload, such as the acidic microenvironment of a tumor.

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

The following examples are presented in order to more fully illustrate some embodiments of the invention. They should in no way be construed, however, as limiting the broad scope of the invention. One skilled in the art can readily devise many variations and modifications of the principles disclosed herein without departing from the scope of the invention.

The invention will now be illustrated by the following non-limiting Examples.

### EXAMPLES

### Materials and Methods

Materials. Tris-HCl buffer was prepared with Trizma base (Sigma Aldrich) and adjusted to pH 7.5 with hydrochloric acid (HCl). Polyuridylic acid (poly-U RNA) was purchased from Sigma Aldrich (P9528). 15 bases Cy3-oligoA was purchased from IDT. Pluronic-F127 for slide coating was purchased from Sigma Aldrich (P2443). Fmoc-L-Lys(Nvoc)-OH was purchased from Iris Biotech (FAA7230). Rhodamine B was purchased from Acros Organics (296570250). Cy5-COOH (MW 483.68 g/mol) was received from Prof. Roey Amir's lab, School of Chemistry, Faculty of Exact Sciences at Tel Aviv University. tRNA was purchased from Sigma-Aldrich (Cat. #10109509001, tRNA 500 mg from baker's yeast). Atto647-UTP was purchased from Jena Bioscience. Tyrosinase extracted from mushroom was purchased from Sigma-Aldrich and labeled at amines using succinimidyl ester functionalized Atto633 labeling kit (Sigma).

Peptide synthesis and purification. All peptides were synthesized at the Blavatnik center for Drug Discovery at Tel Aviv University, by solid phase peptide synthesis (SPPS) using Liberty Blue™ automated microwave peptide synthesizer on Fmoc-Val-Wang resin (0.311 meq/g substitution). Peptide purity was analyzed using LCMS (Waters Autopurification system, collaboration with Blavatnik center, see details below) and analytical reverse-phase high-performance liquid chromatography (RP-HPLC, Thermo Fisher), a Dionex SD Ultimate 3000 UHPLC standard system equipped with a diode-array detection (DAD) Detector. Mobile phases were (A) H2O (0.1% TFA) and (B) MeCN (0.1% TFA), stationary phase used was CS chromatography MultoHigh C18 column (250 mm×4.6 mm, 5 μm particle size and 100 Å pore size, 5861272). Samples were then collected with a preparative RP-HPLC, similar to the analytical system with a Thermo-scientific fraction collector and a corresponding CS chromatography MultoHigh C18 column (250 mm×10 mm, 5 μm particle size and 100 Å pore size, 510117225).

Alanine scanning peptide synthesis and purification. All 14 alanine scan peptides were synthesized in collaboration by Blavatnik center for drug discovery at Tel Aviv University. Peptide synthesis was performed as mentioned above, for V14A peptide Fmoc-Ala-Wang resin (0.380 meq/g substitution) was used. The peptides were analyzed on a Waters Autopurification system analytical module with PDA detector equipped with a Waters Xselect Peptide CSH C18 column (5 μm, 4.6 mm×100 mm). The Autopurification system was connected to a Waters SQD2 MS detector for structural determination. Separation was performed with a gradient elution of 0.1% FA in (i) H2O and (ii) MeCN with Xselect CSH C18 Prep Column (5 μm, 19 mm×250 mm).

Peptide trifluoroacetic acid (TFA) exchange. To remove TFA content which was left following peptide purification, two cycles of TFA exchange (with HCl) were performed. Purified peptide was dissolved in DDW to a final concentration of 2 mM, then 1 M HCL was added at a final concentration of 7.5 mM, after a few moments the samples were freeze-dried. After two cycles of TFA exchange the samples were tested in F-NMR for verification that no TFA was left. F-NMR spectra was collected in D₂O using a Bruker Advance III spectrometer at 400 MHz at the Department of Chemistry NMR Facility at Tel Aviv University.

Formation of compartments, LLPS propensity analysis and phase diagrams. Poly-U was dissolved in ultra-pure water (19.5 mg ml⁻¹) and stored at −20° C. 20 mM VFP-1 stock was prepared in 10 mM Tris buffer and adjusted to pH 7.5. Poly-U was added to peptide solution at varying concentrations to initiate LLPS. Turbidity was immediately visible upon poly-U addition. UV-Vis absorbance spectra were taken using a Synergy H1 microplate reader, at λ=300-700 nm after 30 seconds shaking. Phase diagrams were created based on turbidity measured using 384-well plate at λ=300 nm. To analyze the effect of salt and temperature on LLPS, turbidity was measured at varying concentrations of NaCl (10-40 mM) or varying temperature between 20-60° C. using Synergy H1 microplate reader. Salt was added to samples of 2 mM VFP-1 and 1 mg ml⁻¹ poly-U or 1 mM Nvoc-VFP-1 and 0.5 mg ml⁻¹ poly-U, due to saturation of the Nvoc-VFP-1/poly-U turbidity signal at 2 mM.

Optical microscopy. Optical microscopy images were taken with inverted microscope (OPTIKA Microscopes Italy) with X-LED 8 W lamp and 60× objective using clear glass bottom 96-well plate for microscopy analysis. For optical microscopy analysis of droplet formation by VFP-1/tRNA, 1 mg ml⁻¹ of tRNA was used. 50 μl Samples were analyzed following 2 min incubation at varying temperatures using an orbital mixing-heating incubator (Torrey Pines Scientific Inc).

Dynamic light scattering (DLS). DLS was performed using Malvern Zetasizer ZS instrument equipped with a 633 nm He—Ne laser and aligned for backscattering at 173°. Malvern software was used to analyze inverse Laplace transforms of the intensity autocorrelation functions using the non-4 negatively constrained least squares (NNLS) algorithm to obtain multimodal size distribution data. Samples were prepared and pH-adjusted without poly-U, which was added to initiate LLPS immediately before DLS measurements. Each measurement is a mean of 10 measurements calculated by the Malvern software.

Confocal microscopy. The fluorescence images were obtained using Zeiss Zen 900 confocal microscope with ×20/0.8 NA Plan—Apochromat air objective at 1 AU pinhole. All confocal samples were imaged on top of a Pluronic-F127 coated slide. Cy3 and rhodamine B images were taken with λₑₓ=561 nm lase, λₑₓ=640 nm laser for Cy5.

Fluorescence recovery after photobleaching (FRAP). FRAP analysis measures recovery time of the compartments by using a fluorescent probe and photobleaching a small area in the center of the compartment, and measuring the recovery of fluorescent signal over time in the region of interest. Diffusion constant and t½ were calculated based on the fluorescence recovery plots. All FRAP samples were obtained using 2 mM peptide, 1 mg ml⁻¹ poly-U and 0.04 μM Cy3-oligo-A in 10 mM Tris buffer at pH 7.5. FRAP analysis was performed using Cy3-conjugated oligo-adenosine (Cy3-oligo-A, 15-mer)^{[21]} (λₑₓ=561) with 15× crop magnification (128×128 pixels). Bleach area radius 0.5 μm, using 100% lasers. For Ala scanning FRAP analysis, the following lasers were used for photobleaching: λₑₓ=405 nm, 488 nm, 561 nm, 640 nm. For Nvoc-VFP-1 FRAP analysis, the following lasers were used for photobleaching: λₑₓ=488 nm, 561 nm, and 640 nm, excluding λₑₓ=405 nm to avoid Nvoc photocleavage. Plot exponential fitting and recovery half-life were calculated using OriginLab. Final values were averaged from n=9 (FIG. 3) or n=10 droplets (FIG. 4). Apparent diffusion coefficient (D) of droplets formed by each of the peptides was calculated as:

$${D}_{app} = \frac{{r}^{2}}{t}$$

where t is the recovery time obtained from the recovery plots and r is the radius of the photobleached area.

UV irradiation of Nvoc-VFP-1. UV irradiation was performed using a UV-box equipped with λ=365 nm UV lamp (VILBER-LOURMAT, 6 W). Samples were prepared in 1 ml glass vials and placed inside the box.

Nvoc-VFP-1 cleavage analysis using HPLC. Nvoc-VFP-1 was UV irradiated (λ=365 nm, unless indicated otherwise) for 4 h, 8 h, 12 h, and 24 h. Following each time point, samples were analyzed using analytical RP-HPLC detailed above and % of cleavage was calculated from chromatogram peak integrals using Chromeleon™ software tools.

Dye encapsulation efficiency (EE %) analysis. Nvoc-VFP-1/VFP-1 were dissolved in 10 mM Tris buffer (pH 7.5, 2 mM peptide) and irradiated for 24 h or placed in darkness for 24 h. After 24 h, poly-U (1 mg ml⁻¹) and dye (rhodamine B or Cy5, 5 μM) were added. Droplet samples were centrifuged (1,000 RPM, 10 min) and supernatants were collected for absorbance spectra measurements in triplicates (using 384-well plate). Background measurements of samples without dye and only buffer samples were subtracted from sample measurements. Encapsulation efficiency percentage (EE %) was calculated as:

$$EE \% = \frac{Total dye concentration - Supernatant dye concentration}{Total dye concentration} \times 100$$

Supernatant dye concentration was calculated from calibration curves for each dye (rhodamine B or Cy5) prepared using varying dye concentrations (20, 15, 10, 5, 1, 0.5 μM).

Dye encapsulation in individual droplets. Rhodamine B/Cy5 (0.05 μM), Atto633-tyrosinse (0.7 μM) or Atto647-UTP (1 μM) were added to droplets formed by Nvoc-VFP-1/poly-U or VFP-1/poly-U (using 2 mM peptide and 1 mg ml⁻¹ poly-U, respectively). Droplets encapsulating dye were irradiated using the λₑₓ=405 nm laser, and the fluorescent signal of the dye in the droplets was monitored before and immediately after irradiation using λₑₓ=561/640 nm laser (561 nm for rhodamine B and 640 nm for Cy5, Atto633-tyrosinase and Atto647-UTP). Normalized fluorescence values were obtained by using the average minimal intensity of Nvoc-VFP-1 droplets (n=5 for RhB/Cy5, n=8 for Atto633-tyrosinase/Atto647-UTP).

### Example 1: Viral Factory-Inspired Peptide (VFP-1) Design

The design of the open liquid compartments was inspired by the measles viral factories that are formed by complexation between RNA, the measles virus phosphoprotein (P protein) (FIG. 1) and other viral proteins. The 14-mer viral factory-inspired peptide (VFP-1) LGKSGRLPGKSGRV (SEQ ID NO: 1) was designed based on prevalence of amino acids in the phosphate-binding loop (P-loop) of the P protein, including leucine (Leu), glycine (Gly), proline (Pro), serine (Ser) and valine (Val). Additionally, two arginines (Arg) and two lysines (Lys) were also included within the peptide sequence, since they are present in the P loop at a prevalence of 7% and 11%, respectively. It is hypothesized that Gly would induce flexibility in the disordered peptide chain, which is key for LLPS, while Arg and Lys would promote electrostatic or cation-7r interaction with RNA, and the non-polar amino acids (Val, Leu) would promote hydrophobic interactions between the peptide molecules.

### Example 2: LLPS Propensity of VFP-1 with Poly-U RNA

The LLPS propensity of the VFP-1 peptide and its ability to form liquid compartments were studied by complex coacervation with RNA, using poly-uridylic acid (poly-U) as an RNA model system. VFP-1 forms liquid droplets in the presence of poly-U at low mM peptide concentrations (FIG. 2A). Some LLPS could be seen already at 0.5 mM of peptide and at 0.1 mg/ml of poly-U, and the amount of LLPS increased with an increase in both components.

The ratio of positively-charged residues (from the peptide) to negatively-charged residues (from the poly-U) was calculated as the ratio of VFP-1 total charge to poly-U total charge. taking into account 4 positively-charged residues per each peptide molecule (corresponding to the four basic amino acids) and ˜2940 negatively-charged residues per each poly-U molecule (estimated to have about 2940 bases, on average). It was found that LLPS was obtained for positive to negative charge ratios of 2.4 and above.

Circular dichroism (CD) analysis showed that the peptide is disordered and does not adopt a secondary structure in the presence of poly-U. Tune droplet diameter was achieved by varying the concentration of poly-U, the bulkier of the two components. Dynamic light scattering (DLS) analysis of droplet size distribution accompanied with optical microscopy imaging showed that droplet diameter increased linearly by increasing the concentration of poly-U from 0.1 mg ml⁻¹ to 0.3 mg ml⁻¹ (FIG. 2B). Further increase in poly-U concentration to 0.5 mg ml⁻¹ resulted in formation of droplets with a wide size distribution ranging from 300 nm to 1000 nm. By contrast, varying VFP-1 concentration while keeping a constant poly-U concentration has a minor effect on droplet diameter (FIG. 2C).

Since viral factories sequester both structured and unstructured RNA, an attempt was made to form LLPS between VFP-1 (2 mM) and tRNA (1 mg/ml), as a model for structured RNA. It was found that VFP-1/tRNA LLPS was temperature-dependent, as no droplets were formed at temperatures lower than 40° C. Droplet abundancy increased with increasing temperatures between 40-60° C. (data not shown). These results indicated that VFP-1/RNA LLPS was mediated by complexation of the peptide with partially, or fully, disordered RNA and that unfolding of the tRNA was critical for droplet formation.

### Example 3: Alanine Scanning of VFP-1

To get a better understanding of the role of each amino acid of VFP-1 in LLPS and droplet formation, alanine (Ala) scanning was performed. Each of the amino acids of VFP-1 was substituted with Ala, resulting in 14 sequence variants (Table 1). Following peptide synthesis and purification, the LLPS propensity of each peptide was analyzed at varying poly-U concentrations (0.3, 0.5, 1 mg/ml) (FIG. 3A), where increasing the poly-U concentration increases sample turbidity (corresponding to LLPS and formation of droplets (compartments)). Substituting each of the basic amino acids with Ala (K3A, R6A, K10A, or R13A) completely arrested LLPS and droplet formation (FIG. 3A). This finding correlates with previous work which suggests that peptide/RNA complex coacervation is mainly driven by electrostatic interactions. While all Ala substitutions of non-basic amino acids resulted in droplet formation, substituting specific amino acids with Ala affected LLPS propensity. Specifically, Pro/Ala substitutions (P8A) increases sample turbidity and droplet diameter while milder increase in turbidity is observed for Ser/Ala substitution at position 11 (S11A) and Val/Ala substitution (V14A) (FIG. 3A).

Next, the effect of the amino acid substitutions on the material properties of the droplets was studied. For this, fluorescence recovery after photobleaching (FRAP) analysis of the peptides VFP-1, G5A, P8A, S11A, and V14A, was performed using laser scanning confocal microscopy. Droplets formed by VFP-1/poly-U were highly dynamic, with 96% recovery of fluorescence after photobleaching (FIGS. 3B-3C), with a droplet diffusion rate of about 1.5×10⁻⁷ m²/sec (not shown) and a t_{1/2} of 0.7 sec (FIG. 3D). A decrease in droplet diffusion is observed for P8A, S11A, and V14A (FIGS. 3B-3D) which were about 3-5×10⁻⁸ m²/sec (not shown), with more than a 4-fold increase in t_{1/2} observed for V14A (about 3 sec) compared with VFP-1 (FIG. 3D), while no significant difference in droplet dynamics was observed for G5A. These results suggest that droplet dynamics can be tuned by simple changes to the peptide chemical composition.

TABLE 1
Peptide sequences

| SEQ ID No. | Peptide name | sequence |
|---|---|---|
| 1 | VFP-1 | LGKSGRLPGKSGRV |
| 2 | L1A | AGKSGRLPGKSGRV |
| 3 | G2A | LAKSGRLPGKSGRV |
| 4 | K3A | LGASGRLPGKSGRV |
| 5 | S4A | LGKAGRLPGKSGRV |
| 6 | G5A | LGKSARLPGKSGRV |
| 7 | R6A | LGKSGALPGKSGRV |
| 8 | L7A | LGKSGRAPGKSGRV |
| 9 | P8A | LGKSGRLAGKSGRV |
| 10 | G9A | LGKSGRLPAKSGRV |
| 11 | K10A | LGKSGRLPGASGRV |
| 12 | S11A | LGKSGRLPGKAGRV |
| 13 | G12A | LGKSGRLPGKSARV |
| 14 | R13A | LGKSGRLPGKSGAV |
| 15 | V14A | LGKSGRLPGKSGRA |

### Example 4: Effect of VFP-1 Lysine Modification by Nvoc on LLPS

Based on the above findings, it was hypothesized that the material properties of the compartments can be temporally modulated by controlling peptide charge and polarity using external stimuli. To this end, the side chain of Lys at position 3 was modified with the photocleavable group o-nitroveratryloxycarbonyl (Nvoc) (FIG. 1C, SEQ ID NO: 16), which is cleavable by UV irradiation. Considering that Ala substitution of Lys at this position completely arrested LLPS, it was expected that Nvoc conjugation to Lys would significantly affect LLPS propensity and droplet dynamics. Specifically, it was expected that this hydrophobic aromatic group would promote hydrophobic or π-π interactions between the peptide molecules or between the peptide and RNA, and as a result, decrease droplet dynamics. Thus, light-triggered removal of the Nvoc group from compartments that are formed by the Lys-modified peptide (Nvoc-VFP-1) should increase peptide polarity, reduce peptide-peptide contacts, promote electrostatic interactions between the peptide and poly-U, and increase droplet dynamics, as would be reflected in the diffusion and mobility of peptide/RNA.

Following synthesis and purification of Nvoc-VFP-1, the effect of temperature and salt on formation of liquid droplets by VFP-1/RNA compared to Nvoc-VFP-1/RNA was first analyzed. Addition of NaCl at concentrations ranging between 10-40 mM resulted in concentration-dependent inhibition of VFP-1/RNA LLPS, where sample turbidity gradually decreased at increasing NaCl concentrations and a complete arrest of droplet formation was observed at 40 mM NaCl (FIG. 4A). In contrast, salt had a minor effect on Nvoc-VFP-1/RNA droplet formation, as 40 mM NaCl resulted in only a 17% decrease in sample turbidity, and no change in droplet abundance was observed by optical microscopy. These results correlate with the findings from the Ala scanning analysis, further suggesting that the main driving force of VFP-1/RNA LLPS is electrostatic interactions, whereas Nvoc-VFP-1/RNA droplets are formed by additional modes of interaction such as π-π, cation-π, or hydrophobic interactions. Increasing the temperature of the sample had a negligible effect on droplet formation by both peptides (FIG. 4B), ruling out contribution of hydrogen bonding in LLPS of either VFP-1/RNA or Nvoc-VFP-1/RNA.

Nvoc cleavage from the peptide as a function of UV irradiation (λ=365 nm) was analyzed using HPLC. This analysis confirmed that the Nvoc group was ˜95% cleaved from the peptide after irradiation for 24 h. Next, the effect of compartment irradiation on the material properties of individual droplets was evaluated using FRAP analysis, where droplets were measured following varying irradiation time. First, it can be seen from time point 0 in FIGS. 4C-4E that the recovery of the Nvoc-modified peptide after photobleaching was indeed very slow, as expected. This analysis further showed that the total recovery of fluorescent signal after photobleaching depended on the irradiation time, where 6 hours of irradiation increased the total recovery by 16% compared with unirradiated compartments (FIGS. 4C-4E). Moreover, controlling the irradiation time resulted in tunable diffusion coefficient (D) and t_{1/2} (FIGS. 4D-4E) with an about 6-fold increase in droplet diffusion (about 7×10⁻⁹ to about 4.2×10⁻⁸ m²/sec), and an >6-fold decrease in t_{1/2} (from 15.55 sec to 2.45 sec) following irradiation for 18 hours (FIG. 4E). These findings suggested that increasing droplet polarity by cleavage of the hydrophobic aromatic Nvoc group from the peptide building block increased droplet dynamics. Thus, the material properties of compartments can be temporally regulated by light-controlled changes to the chemical composition of the peptide building block.

### Example 5: Effect of Anionic Polymer Structure on LLPS

An experiment testing the effect of various anionic polymers on LLPS was conducted as follows: 2 mM of peptide were incubated with an anionic polymer at several different conditions, as follows: 10 mM Tris-HCl (pH=7.5), Tris-HCl+20 mM NaCl, Tris-HCl+0.1 mM EDTA, or Tris-HCl+NaCl+EDTA, each at either 25° C. or 70° C. Two peptides were used: an unprotected VFP-1 peptide (L7A, SEQ ID NO: 8, which only differs from the original VFP-1 by an alanine substitution of leucine at position 7, and which was found to have a similar activity), and an Nvoc-modified VFP-1 (SEQ ID NO: 16). The anionic polymers were three ssDNA molecules and one tRNA molecule. The ssDNA molecules were: 1. tDNAh (SEQ ID NO: 17), which has the sequence of a natural human tRNA (URS00006D30C1_9606 at the RNA central database) and based on the sequence is predicted to form four stem and loop structures; 2. tDNAd (SEQ ID NO: 18), a designed sequence with a similar length and GC % to tDNAh, also predicted to form four stem and loop structures but with a higher propensity; and 3. tDNA[oligoA](SEQ ID NO: 19), which comprises the nucleotides at positions 9-45 (37 nu, 50% of the complete sequence) of tDNAd with the rest of the 37 nucleotides replaced with poly A, and is expected to form a less ordered structure; each used at 0.05 mM. The tRNA (Sigma, Cat. #10109509001), was used at 0.5 mg/ml with the Nvoc-protected peptide, or at 1 mg/ml with the unprotected L7A peptide. The ssDNA sequences in this example were designed by the aid of software: for creating random sequences—Random DNA Sequence Generator from University of California, Riverside was used, and for analysis of predicted structure and melting temperature—the OligoAnalizer™ Tool from IDT was used.

The results (not shown) indicated that the Nvoc protected peptide was able to form an LLPS-based compartment at all conditions, i.e., regardless of salt concentration and temperature) and with all anionic polymers, i.e., regardless of the level of order/structure. In contrast, the L7A non-protected peptide showed LLPS at 25° C. only with tDNAh, but not with either the partially unordered tDNA[oligoA] or with the highly ordered tRNA and the designed tDNAd. At an elevated temperature of 70° C. the L7A peptide showed LLPS with all anionic polymers except for the partly unordered tDNA[oligoA]. This was surprising since it was assumed that the ordered polymers need a higher temperature for the secondary structures to open in order to form LLPS, while unordered structures are already open and therefore more amenable to LLPS. Regarding the LLPS with tDNAh at 25° C., it may be possible that while the corresponding tRNA sequence folds naturally, the ssDNA tDNAh does not fold properly in the in vitro conditions, which allows for LLPS.

TABLE 2
ssDNA sequences

| SEQ ID | ssDNA name | sequence |
|---|---|---|
| 17 | tDNAh | TTGGTTACGTCCTCACTTAGGTGAGGAGAATGGAAACGCACCA |
| | | TTGAATTATGGTTACTTTTACCATTAACCAA |
| 18 | tDNAd | TGCATACGGTCCTCACTTAGGTGAGGAGAATGGAAACGCACCA |
| | | TTATTATGGTTACTTTTACCATGAGTATGCA |
| 19 | tDNA[oligoA] | AAAAAAAAGTCCTCACTTAGGTGAGGAGAATGGAAACGCACCA |
| | | TTAAAAAAAAAAAAAAAAAAAAAAAAAAAAA |

One of the conclusions from this example is that a system including an Nvoc-protected peptide together with a structured, or a partially structured RNA, may be useful for control of payload entrapment and release, since the Nvoc-protected peptide forms LLPS with a structured RNA, and therefore is able to entrap a payload (as shown in Example 6 below), and once the Nvoc is removed (by a UV light), the compartment disassembles and the payload is released.

### Example 6: Effect of Compartment Composition on Payload Dynamics

It was next investigated whether the temporal control over the peptide chemical composition and in turn droplet dynamics could be leveraged to regulate payload encapsulation and release from the compartments. To this end, the encapsulation of a fluorescent payload by the compartment following irradiation was analyzed using a hydrophilic dye, rhodamine B (RhB), and a hydrophobic dye, Cyanine-5 (Cy5). The calculated RhB encapsulation efficiency (EE %) of compartments formed by Nvoc-VFP-1 was 88% compared to 40% of VFP-1 compartments (FIG. 5A). This suggested that the Nvoc group promotes RhB encapsulation either by direct binding through hydrophobic or aromatic interactions, or indirectly, by inducing an overall hydrophobic microenvironment within the condensate (compartment) which facilitates the recruitment of the dye. Following irradiation of the Nvoc-VFP-1 compartments, the EE % of RhB decreases by 40% (FIG. 5A). Irradiation of unmodified VFP-1 compartments had no significant effect on the EE % of RhB. These results suggested that the encapsulation of RhB can be temporally controlled. When studying an additional, hydrophobic, fluorescent payload system using Cy5, similar trends were found. However, the EE % of Cy5 by Nvoc-VFP-1 compartments was significantly higher (96%) than that of RhB (FIG. 5D), and the decrease in the EE % following irradiation was much lower (about 6%). The large difference between the % EE of RhB and that of Cy5 implies that the latter is not mediated by hydrophobic or aromatic interactions but rather by other modes i.e., electrostatic interactions with the basic side chains of the peptide.

Next, spatiotemporal analysis of RhB and Cy5 partitioning and release from droplets was performed using confocal microscopy. Individual droplets formed by Nvoc-VFP-1 or VFP-1 were selectively excited using λₑₓ=405 nm laser. Excitation at this wavelength is expected to induce Nvoc cleavage but does not lead to photobleaching of RhB nor Cy5, as confirmed by analyzing the fluorescent signal of the dyes in VFP-1 droplets before and after irradiation (RhB: FIGS. 5B-6C; Cy5: FIGS. 5E-6F). Immediately after irradiating the Nvoc-VFP-1 compartments containing RhB or Cy5, the fluorescent signal of the dye dropped drastically (FIG. 5C, 5E). It is noted that the drop in fluorescence is due to cleavage of the Nvoc, which is released faster at the higher wavelength used in this experiment. A partial increase in RhB signal was observed in the droplet over time, the finite recovery reaching about 60% of the initial intensity, suggesting that RhB was released from the compartment. In contrast, no effect on the fluorescent signal of RhB in VFP-1 compartments was observed following irradiation. The finite fluorescent intensity of Cy5 in the Nvoc-VFP-1 droplets reaches about 88% following irradiation, which corresponded to the moderate change in % EE of the dye in VFP-1 compartments following irradiation.

In addition, the partitioning and release of the enzyme tyrosinase and uridine-triphosphate (UTP) as model systems for enzymes and nucleotides, which are abundant in viral factories, were analyzed. Similar to Cy5 release, a sharp decrease in the fluorescence of Atto633-labeled tyrosinase and Atto647-labeled UTP was observed exclusively in Nvoc-VFP-1 droplets, but not in VFP-1 droplets, following irradiation (data not shown). In addition, rapid fusion of neighboring droplets was observed only for Nvoc-VFP-1 and not for VFP-1 droplets, immediately after irradiation.

These results suggest that it is possible to spatially and selectively control the chemical composition of the compartments, and through light stimuli create a photochemical reaction that might allow real-time release of comparted molecules to the droplet surroundings.

1.-44. (canceled)

45. A composition comprising a liquid-liquid phase separation (LLPS)-based compartment comprising a cationic peptide and an anionic polymer.

46. The composition of claim 45, wherein the cationic peptide comprises at least one aromatic amino acid, and/or at least one amino acid modified by a hydrophobic and/or an aromatic cleavable group.

47. The composition of claim 45, wherein the cationic peptide comprises at least one positively-charged amino acid and/or at least one hydrophobic amino acid.

48. The composition of claim 45, wherein the cationic peptide comprises at least about 10% positively-charged amino acids.

49. The composition of claim 47, wherein the positively-charged amino acids are selected from lysine (Lys), arginine (Arg), and histidine (His), and/or the hydrophobic amino acid is selected from the group consisting of leucine (Leu), valine (Val), Alanine (Ala), isoleucine (Ile), and methionine (Met).

50. The composition of claim 46, wherein the aromatic amino acid is selected from the group consisting of phenylalanine (Phe), tyrosine (Tyr), Histidine (His), and tryptophan (Trp).

51. The composition of claim 45, wherein the cationic peptide further comprises glycine (Gly), proline (Pro), Serine (Ser), and/or threonine (Thr).

52. The composition of claim 45, wherein the cationic peptide comprises the amino acids Arg or Lys, and Leu, Gly, Pro, Ser, and Val.

53. The composition of claim 45, wherein the cationic peptide comprises an amino acid sequence of a protein phosphate-binding loop (P-loop).

54. The composition of claim 45, wherein the cationic peptide comprises a sequence selected from the the group consisting of sequences SEQ ID NOs: 1, 2, 3, 5, 6, 8, 9, 10, 12, 13, and 15.

55. The composition of claim 46, wherein the cleavable group is selected from the group consisting of a photocleavable group, a chemically cleavable group, and an enzymatically cleavable group.

56. The composition of claim 55, wherein the cleavable group is a photocleavable group selected from the group consisting of ortho-nitrobenzyl (o-nitrobenzyl, ONB), o-nitroveratryloxycarbonyl (Nvoc), and a BODIPY (C₉H₇BN₂F₂ or 4,4-difluoro-4-bora-3a,4a-diaza-s-indacene)-derived photocleavable protecting group.

57. The composition of claim 46, wherein the at least one cleavable group is bound to a positively-charged amino acid.

58. The composition of claim 46, wherein the cationic peptide comprises a sequence according to SEQ ID NO: 16, including a modification of lysine at position 3 of SEQ ID NO: 1 by Nvoc.

59. The composition of claim 45, wherein the anionic polymer is a nucleic acid.

60. The composition of claim 59, wherein the nucleic acid is RNA.

61. The composition of claim 45, further comprising a payload.

62. The composition of claim 61, wherein the payload is hydrophobic.

63. A method of delivery of a payload to a cell by using the composition of claim 45.

64. A method of preparing a liquid-liquid phase separation (LLPS)-based compartment, the method comprising mixing a cationic cationic peptide, an anionic polymer, and optionally a payload, wherein the cationic peptide comprises at least one aromatic amino acid, or at least one amino acid modified by a hydrophobic and/or an aromatic cleavable group.
